(19) United States
(12) Patent Application Publication
(10) Pub. No.: US 2010/0266681 A1
(43) Pub. Date: Oct. 21, 2010

(54) FATTY ACID ALCOHOLS

(76) Inventor: Anne Kristin Holmeide, Oslo (NO)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/447,971

(22) PCT Filed: Nov. 2, 2007
(86) PCT No.: PCT/IB07/04590

§ 371 Date: Jun. 25, 2010

Related U.S. Application Data
(60) Provisional application No. 60/856,267, filed on Nov. 3, 2006.
(60) Provisional application No. 60/856,268, filed on Nov. 3, 2006.

(30) Foreign Application Priority Data
Nov. 3, 2006 (SE) 0602352-7

(51) Int. Cl. A61K 31/045; A61K 9/48; A61K 9/50; A61K 31/22; A61K 31/225; A61K 31/232; C07C 69/587; C07C 69/24; C07C 69/145; C07D 295/037; C08B 37/08; A61P 3/06; A61K 8/34

(52) U.S. Cl. 424/451; 514/739; 424/490; 514/546; 514/547; 514/549; 554/224; 560/201; 560/265; 544/358; 536/20

(57) ABSTRACT

The present invention relates to a lipid composition comprising at least omega-3 polyunsaturated alcohols, or pro-drugs thereof, which omega-3 polyunsaturated alcohols, or pro-drugs thereof, comprising at least (all-Z)-5,8,11,14,17-eicosapentaen-1-ol, or pro-drug thereof, and (all-Z)-4,7,10,13,16,19-docosahexaen-1-ol, or pro-drug thereof, and their use as a pharmaceutical, in particular for the treatment of elevated triglyceride levels. The invention also relates to methods for the preparation of these pro-drugs from marine oils.

### FIELD OF THE INVENTION

The present invention relates to a lipid composition comprising at least omega-3 polyunsaturated alcohols, or pro-drugs thereof, which omega-3 polyunsaturated alcohols, or pro-drugs thereof, comprise at least one of (all-Z) 5,8,11,14,17-eicosapentaen-1-ol, or a pro-drug thereof, (all-Z)-4,7,10,13,16,19-docosahexaen-1-ol, or a pro-drug thereof, and (all-Z)-9,12,15-octadecatrien-1-ol, or a pro-drug thereof, and their use as pharmaceuticals for reducing elevated triglyceride levels in humans and animals, including non-human mammals. The present invention also relates to lipid compositions for as a cosmetic skin preparation. The present invention also relates to methods for the preparation of these polyunsaturated alcohols, or pro-drugs thereof, from marine oils. The invention further relates to novel omega-3 polyunsaturated pro-drugs and salts of said pro-drugs. Salts of the pro-drugs can be, for example, salts of hemisuccinate esters.

### BACKGROUND OF THE INVENTION

Dietary omega-3 polyunsaturated fatty acids like (all-Z)-eicosapentaenoic acid (EPA) and (all-Z)-docosahexaenoic acid (DHA), have effects on diverse physiological processes impacting normal health and chronic diseases, such as the regulation of plasma lipid levels, cardiovascular and immune functions, insulin action and neural development and visual function. Highly purified polyunsaturated fatty acids in the form of ethyl esters have been shown to efficiently reduce elevated levels of triglycerides in humans.

One such form of omega-3 fatty acids is a concentrate of omega-3, long chain, polyunsaturated fatty acids from fish oil containing DHA and EPA as ethyl esters, described, for example, in U.S. Pat. Nos. 5,502,077; 5,656,667; and 5,698,594, each incorporated herein by reference, and is sold under the trademark Omacor® or Lovaza®. Specifically, a fatty acid composition containing a high concentration, of at least 80% by weight, of omega-3 fatty acids as ethyl esters, where EPA ethyl ester and DHA ethyl ester are present in relative amounts of 1:2 to 2:1, and constitute about at least 75% of the total fatty acids in the composition, has shown surprisingly advantageous effects on several risk factors for cardiovascular diseases, especially exhibiting beneficial effects on hypertriglyceridemia, mild hypertension, and on the coagulation factor VII phospholipid complex activity. Such compounds, including Omacor® and Lovaza®, lower serum LDL-cholesterol, increase serum HDL-cholesterol, lower serum triglycerides, lower systolic and diastolic blood pressure and the pulse rate, and lower the activity of the blood coagulation factor VII-phospholipid complex. EPA and DHA have been shown to operate synergistically. Additionally, at least one advantage of a fatty acid composition described herein is that they are very well tolerated, not giving rise to any severe side effects.

### SUMMARY OF THE INVENTION

The aim of the present invention is to provide a new lipid composition comprising omega-3 polyunsaturated alcohols, or pro-drugs thereof, having therapeutic activity.

The present invention includes a number of aspects. Some of these aspects are:
1. A novel lipid composition, comprising omega-3 polyunsaturated alcohols.
2. A novel lipid composition, comprising pro-drugs of omega-3 polyunsaturated alcohols.
3. A novel lipid composition, comprising a combination of (all-Z)-5,8,11,14,17-eicosapentaen-1-ol and (all-Z)-4,7,10,13,16,19-docosahexaen-1-ol.
4. A novel lipid composition, comprising a combination of a pro-drug of (all-Z)-5,8,11,14,17-eicosapentaen-1-ol and a pro-drug of (all-Z)-4,7,10,13,16,19-docosahexaen-1-ol.
5. A lipid composition for use as a medicament, a pharmaceutical and/or a supplement.
6. A pharmaceutical composition for the treatment of elevated triglyceride levels comprising at least omega-3 polyunsaturated alcohols, wherein preferably at least 70% of the omega-3 polyunsaturated alcohols comprises (all-Z)-5,8,11,14,17-eicosapentaen-1-ol and (all-Z)-4,7,10,13,16,19-docosahexaen-1-ol.
7. Use of the lipid composition for the manufacture of a medicament, a pharmaceutical and/or a food or nutritional supplement, for the treatment and/or prevention of hypertriglyceridemia, dyslipidemia, hypertension, hypercholesteremia, post-myocardial infarction (MI) or depression, heart failure, cardiac arrhythmias or atrial fibrillation, IgA Nephropathy, vascular diseases and/or atherosclerotic diseases.
8. Use of the lipid composition comprising at least (all-Z)-5,8,11,14,17-eicosapentaen-1-ol and (all-Z)-4,7,10,13,16,19-docosahexaen-1-ol for treatment of hyperlipidemic conditions, preferably for treatment of hypertriglyceridemia (HTG).
9. A method for the treatment and/or prevention of the diseases or conditions described herein.
10. A process for preparing high concentrates of omega-3 polyunsaturated alcohols, or pro-drugs thereof, from marine oils.
11. Novel pro-drugs of omega-3 polyunsaturated alcohols.

According to a first aspect of the invention, the present invention relates to a lipid composition comprising at least omega-3 polyunsaturated alcohols, wherein the omega-3 polyunsaturated alcohols comprise at least (all-Z)-5,8,11,14,17-eicosapentaen-1-ol and (all-Z)-4,7,10,13,16,19-docosahexaen-1-ol.

In an exemplary embodiment of the invention, a lipid or pharmaceutical composition comprises alcohols of the omega-3 fatty acid ethyl ester compositions described in the U.S. Pat. Nos. 5,502,077; 5,656,667; and 5,698,594, such as for instance a lipid composition comprising:

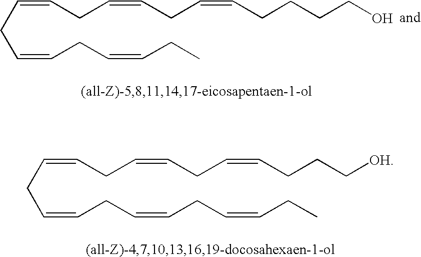

According to a second aspect of the invention, the present invention relates to a lipid composition comprising at least a pro-drug of omega-3 polyunsaturated alcohols, wherein the pro-drugs of omega-3 polyunsaturated alcohols comprise at least pro-drugs of (all-Z)-5,8,11,14,17-eicosapentaen-1-ol and pro-drugs of (all-Z)-4,7,10,13,16,19-docosahexaen-1-ol.

In an exemplary embodiment, the invention relates to a lipid composition, wherein a pro-drug of (all-Z) 5,8,11,14,17-eicosapentaen-1-ol is a compound of formula (III),

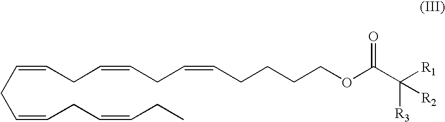

wherein,
R₁, R₂, and R₃ are chosen from:

a hydrogen atom,

a C₁-C₂₂ alkyl, and

a C₁-C₂₂ alkenyl with 1 to 6 double bonds in Z or E configuration, wherein the alkyl and alkenyl groups are optionally substituted, or a salt thereof.

In an exemplary embodiment, the invention relates to a lipid composition, wherein a pro-drug of (all-Z)-4,7,10,13,16,19-docosahexaen-1-ol is a compound of formula (IV):

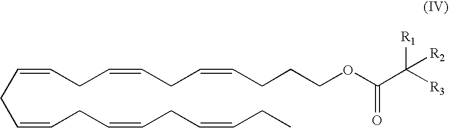

wherein

R₁, R₂, and R₃ are chosen from:

a hydrogen atom,

a C₁-C₂₂ alkyl, and

a C₁-C₂₂ alkenyl with 1 to 6 double bonds in Z or E configuration, wherein the alkyl and alkenyl groups are optionally substituted, or a salt thereof.

In an exemplary embodiment, the invention relates to a lipid composition, wherein a pro-drug of (all-Z)-9,12,15-octadecatrien-1-ol is a compound of formula (V):

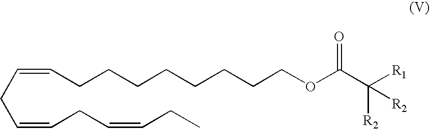

wherein R₁, R₂, and R₃ are chosen from:

a hydrogen atom,

a C₁-C₂₂ alkyl, and

a C₁-C₂₂ alkenyl with 1 to 6 double bonds in Z or E configuration, wherein the alkyl and alkenyl groups are optionally substituted, or a salt thereof.

In an exemplary embodiment, the invention relates to a lipid composition, wherein a pro-drug of (all-Z) 5,8,11,14,17-eicosapentaen-1-ol is chosen from (5Z,8Z,11Z,14Z,17Z)-eicosapentaen-1-yl pivaloate, (5Z,8Z,11Z,14Z,17Z)-eicosapentaen-1-yl hemisuccinate, and [(all-Z)-5,8,11,14,17-eicosapentaen-1-yl] (all-Z)-4,7,10,13,16-eicosapentaenoate.

In an exemplary embodiment, the invention relates to a lipid, wherein a pro-drug of (all-Z)-4,7,10,13,16,19-docosahexaen-1-ol is chosen from (4Z,7Z,10Z,13Z,16Z,19Z)-docosahexaen-1-yl pivaloate, (4Z,7Z,10Z,13Z,16Z,19Z)-docosahexaen-1-yl hemisuccinate, and [(all-Z)-4,7,10,13,16,19-docosahexaen-1-yl] (all-Z)-3,6,9,12,15,18-docosahexaenoate.

In an exemplary embodiment, the invention relates to a lipid, wherein a pro-drug of (all-Z)-9,12,15-octadecatrien-1-ol is chosen from (all-Z)-9,12,15-octadecatrien-1-yl pivaloate, (all-Z)-9,12,15-octadecatrien-1-yl hemisuccinate, and [(all-Z)-9,12,15-octadecatrien-1-yl] (all-Z)-9,12,15-octadecatrienoate.

According to a third aspect of the invention, the present invention relates to a pharmaceutical composition for the treatment of elevated triglyceride levels comprising at least omega-3 polyunsaturated alcohols in a concentration of at least 80% by weight of the total lipid content of the composition, and wherein a combination of (all-Z)-5,8,11,14,17-eicosapentaen-1-ol and (all-Z)-4,7,10,13,16,19-docosahexaen-1-ol is present in a concentration of at least 70% of the omega-3 polyunsaturated alcohols and wherein the weight ratio of (all-Z)-5,8,11,14,17-eicosapentaen-1-ol:(all-Z)-4,7,10,13,16,19-docosahexaen-1-ol is from 1:3 to 3:1.

According to a fourth aspect of the invention, the present invention relates to a use of a lipid composition for the manufacture of a medicament, a pharmaceutical and/or a food or nutritional supplement, for the treatment and/or prevention of hypertriglyceridemia (HTG), dyslipidemia, hypertension, hypercholesteremia, post-myocardial infarction (MI) or depression, heart failure, cardiac arrhythmias or atrial fibrillation, vascular diseases and/or atherosclerotic diseases.

In an exemplary embodiment, the present invention relates to a use of a lipid composition for the manufacture of a medicament, a pharmaceutical and/or a food or nutritional supplement, for the prevention and/or treatment of hyperlipidemic conditions.

According to a fifth aspect of the invention, the present invention relates to a method of treatment and/or prevention of hypertriglyceridemia (HTG), dyslipidemia, hypertension, hypercholesteremia, post-myocardial infarction (MI) or depression, heart failure, cardiac arrhythmias or atrial fibrillation, high risk patients with homeostasis, IgA Nephropathy, vascular diseases and/or atherosclerotic diseases, wherein a therapeutically effective amount of the lipid composition is administered to a human or an animal.

In an exemplary embodiment, the present invention relates to a method for reducing abnormal triglyceride levels in a patient, preferably reducing triglyceride levels of about 200 to about 499 mg/dl, wherein a therapeutically effective amount of the lipid composition to a human or an animal.

According to a sixth aspect of the invention, the present invention relates to a process for manufacture of a lipid composition as described herein.

A seventh aspect of the invention relates to a compound of formula (III):

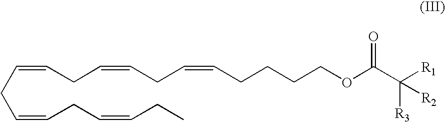

wherein,
R₁, R₂, and R₃ are chosen from:

a hydrogen atom,

a C₁-C₂₂ alkyl, and

a C₁-C₂₂ alkenyl with 1 to 6 double bonds in Z or E configuration, wherein the alkyl and alkenyl groups are optionally substituted, or a salt thereof;

a compound of formula (IV):

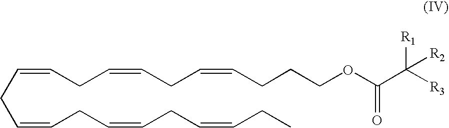

wherein
R₁, R₂, and R₃ are chosen from:

a hydrogen atom,

a C₁-C₂₂ alkyl, and

a C₁-C₂₂ alkenyl with 1 to 6 double bonds in Z or E configuration, wherein the alkyl and alkenyl groups are optionally substituted, or a salt thereof; and

a compound of formula (V):

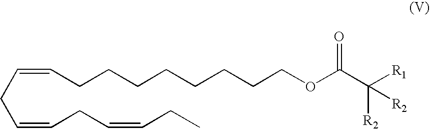

wherein R₁, R₂, and R₃ are chosen from:

a hydrogen atom,

a C₁-C₂₂ alkyl, and

a C₁-C₂₂ alkenyl with 1 to 6 double bonds in Z or E configuration, wherein the alkyl and alkenyl groups are optionally substituted, or a salt thereof.

In an exemplary embodiment, the invention relates to pivaloate esters of omega-3 polyunsaturated compounds chosen from:

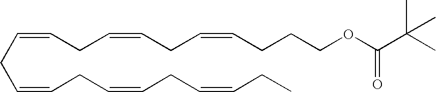

(all-Z)-4,7,10,13,16,19-docosahexaen-1-yl pivaloate;

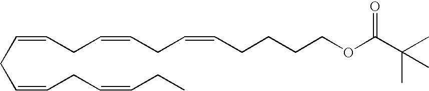

(all-Z)-5,8,11,14,17-eicosapentaen-1-yl pivaloate; and

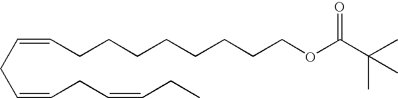

(all-Z)-9,12,15-octadecatrien-1-yl pivaloate.

In another exemplary embodiment, the invention relates to hemisuccinate esters of omega-3 polyunsaturated compounds, or salts thereof chosen from:

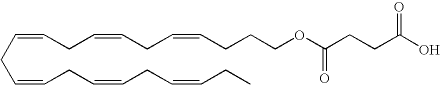

(all-Z)-4,7,10,13,16,19-docosahexaen-1-yl hemisuccinate, or a salt thereof;

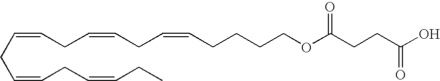

(all-Z)-5,8,11,14,17-eicosapentaen-1-yl hemisuccinate, or a salt thereof; and

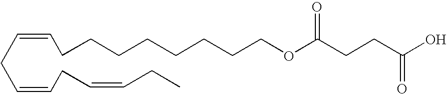

(all-Z)-9,12,15-octadecatrien-1-yl hemisuccinate, or a salt thereof.

In another exemplary embodiment, the invention relates to omega-3 polyunsaturated compounds chosen from:

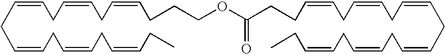

[(all-Z)-4,7,10,13,16,19-docosahexaen-1-yl] (all-Z)-3,6,9,12,15,18-docosahexaenoate;

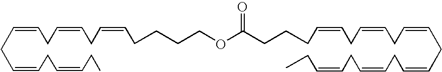

[(all-Z)-5,8,11,14,17-eicosapentaen-1-yl] (all-Z)-4,7,10,13,16-eicosapentaenoate; and

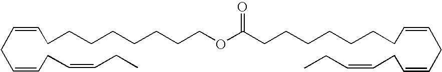

[(all-Z)-9,12,15-octadecatrien-1-yl] (all-Z)-9,12,15-octadecatrienoate.

### DETAILED DESCRIPTION OF THE INVENTION

A number of clinical studies report that mixtures of very long chain alcohols (C24-C34), like octacosanol or policosanol, lower LDL cholesterol and raise HDL cholesterol. No toxicity has been observed except in subjects with inherited metabolic defects, and some evidence suggests that long chain alcohols may improve aspects of muscular performance. Moreover, it is proposed that the alcohols are pro-drugs of the long chain fatty acids generated in vivo.

Evidence suggests that long chain fatty acids and alcohols of up to at least C24 are reversibly interconverted. Enzyme systems exist in the liver, fibroblasts, and the brain that convert fatty alcohols to fatty acids. In some tissues, fatty acids can be reduced back to alcohols. The carboxylic acid functional group is important for targeting binding, but this ionisable group may hinder the drug from crossing the cell membranes of the gut wall. Due to this, carboxylic acids functional groups are often protected as esters. The ester is less polar than the carboxylic acid and can cross the fatty cell membranes. Once in the bloodstream, it can be hydrolysed back to the free carboxylic acid by enzyme esterases in the blood.

It may be possible that the plasma enzymes do not hydrolyse these esters fast enough, and that the conversion of ester to free carboxylic acid predominantly takes place in liver. Ethyl esters of polyunsaturated fatty can also be hydrolysed to free carboxylic acids in vivo.

Thus, there is a need for new pro-drugs of polyunsaturated fatty acids having improved therapeutic activity, increased bioavailability, and ability to cross cell membranes.

The present invention meets these needs with a lipid composition comprising omega-3 polyunsaturated alcohols, or pro-drugs thereof, which omega-3 polyunsaturated alcohols, or pro-drugs thereof, comprise at least (all-Z)-5,8,11,14,17-eicosapentaen-1-ol, or pro-drug thereof, and (all-Z)-4,7,10,13,16,19-docosahexaen-1-ol, or pro-drug thereof.

In exemplary embodiments, the lipid compositions according to the invention comprise alcohols of the omega-3 fatty acids, as described in U.S. Pat. Nos. 5,502,077; 5,656,667; and 5,698,594.

Moreover, it has been surprisingly found that a lipid composition comprising at least the combination of the omega-3 polyunsaturated alcohols of the formula (I):

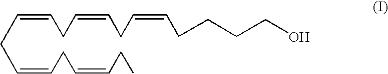

and of the formula (II):

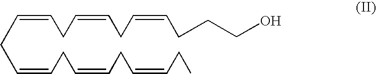

is suitable and may be useful for achieving the desired pharmaceutical activity.

Among possible pro-drugs of polyunsaturated omega-3 alcohols according to the invention, are pro-drugs of formula (III) and (IV):

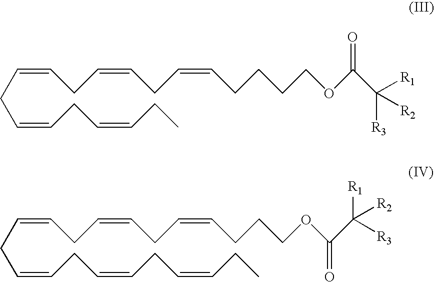

wherein, R₁, R₂, and R₃ are chosen from:

a hydrogen atom,

a C₁-C₂₂ alkyl, and

a C₁-C₂₂ alkenyl with 1 to 6 double bonds in Z or E configuration, wherein the alkyl and alkenyl groups are optionally substituted, or a salt thereof.

In an exemplary embodiment of the invention, the lipid composition comprises at least a pro-drug of an omega-3 polyunsaturated alcohol of formula (VI):

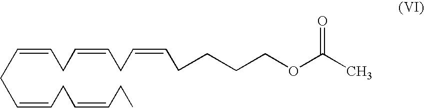

a pro-drug of an omega-3 polyunsaturated alcohol of formula (VII):

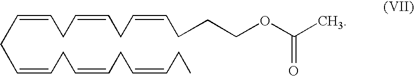

In an exemplary embodiment of the invention, the lipid composition comprises at least a pro-drug of an omega-3 polyunsaturated alcohol of formula (VIII):

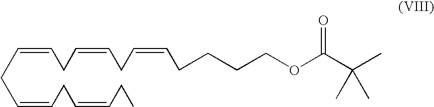

a pro-drug of an omega-3 polyunsaturated alcohol of formula (IX)

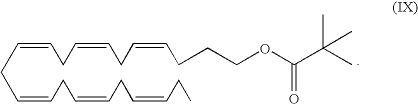

In an exemplary embodiment of the invention, the lipid composition comprises at least a pro-drug of an omega-3 polyunsaturated alcohol of formula (X):

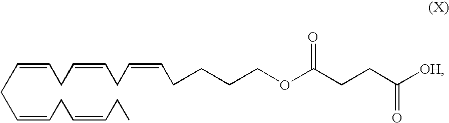

or a salt thereof,
and
a pro-drug of an omega-3 polyunsaturated alcohol of formula (XI)

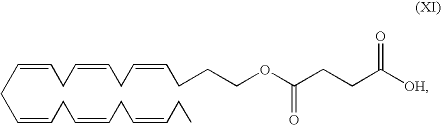

or a salt thereof.

Another lipid composition according to the invention includes omega-3 polyunsaturated alcohols, or pro-drugs thereof, in a concentration of least 30% by weight as compared to the total lipid content of the composition, preferably at least 50% by weight, more preferably at least 60%, still more preferably a least 70% by weight, or most preferably at least 80% by weight, or even at least 90% by weight.

The omega-3 polyunsaturated alcohols, or pro-drugs thereof, in the lipid composition comprise least about 20% by weight of (all-Z)-5,8,11,14,17-eicosapentaen-1-ol, or a pro-drug thereof, and (all-Z)-4,7,10,13,16,19-docosahexaen-1-ol, or a pro-drug thereof, more preferably at least 60% by weight, still more preferably least about 70% by weight, most preferably at least about 80% by weight. In an exemplary embodiment, the omega-3 polyunsaturated alcohols comprise about 84% by weight of (all-Z)-5,8,11,14,17-eicosapentaen-1-ol and (all-Z)-4,7,10,13,16,19-docosahexaen-1-ol.

In an exemplary embodiment of the invention, the omega-3 polyunsaturated alcohols, or pro-drugs thereof, in the lipid composition comprise at least about 20% to 30% by weight of (all-Z)-5,8,11,14,17-eicosapentaen-1-ol, or a pro-drug thereof, and (all-Z)-4,7,10,13,16,19-docosahexaen-1-ol, or a pro-drug thereof. This may, for instance, be the case when the raw material, or crude oil, is a cod-liver oil or a sardine oil.

Further, preferably the omega-3 polyunsaturated alcohols, or pro-drugs thereof, comprise about 5% to about 95% by weight of (all-Z)-5,8,11,14,17-eicosapentaen-1-ol, or a pro-drug thereof, of the total lipid content in the composition, more preferably, about 40% to about 55% by weight of (all-Z)-5,8,11,14,17-eicosapentaen-1-ol, or a pro-drug thereof. Moreover, preferably the omega-3 polyunsaturated alcohols, or pro-drugs thereof, comprise about 5% to about 95% by weight of (all-Z)-4,7,10,13,16,19-docosahexaen-1-ol, or a pro-drug thereof, of the total lipid content in the composition, and more preferably the omega-3 polyunsaturated alcohols, or pro-drugs thereof, comprise about 30% to about 60% by weight of (all-Z)-4,7,10,13,16,19-docosahexaen-1-ol, or a pro-drug thereof.

In an exemplary embodiment of the invention, the omega-3 polyunsaturated alcohols, or pro-drugs thereof, comprise about 43 to 50% of (all-Z)-5,8,11,14,17-eicosapentaen-1-ol and 35 to 40% of (all-Z)-4,7,10,13,16,19-docosahexaen-1-ol, by weight of the total lipid content in the composition.

In an exemplary embodiment of the invention, the omega-3 polyunsaturated alcohols, or pro-drugs thereof, may comprise (all-Z)-5,8,11,14,17-eicosapentaen-1-ol, or a pro-drug thereof and (all-Z)-4,7,10,13,16,19-docosahexaen-1-ol, or a pro-drug thereof, in a weight ratio of (all-Z)-5,8,11,14,17-eicosapentaen-1-ol:(all-Z)-4,7,10,13,16,19-docosahexaen-1-ol from 99:1 to 1:99, more preferably in a weight ratio of (all-Z)-5,8,11,14,17-eicosapentaen-1-ol:(all-Z)-4,7,10,13,16,19-docosahexaen-1-ol from 10:1 to 1:10, still more preferably in a weight ratio of (all-Z)-5,8,11,14,17-eicosapentaen-1-ol:(all-Z)-4,7,10,13,16,19-docosahexaen-1-ol from 5:1 to 1:5, and most preferred in a ratio of (all-Z)-5,8,11,14,17-eicosapentaen-1-ol:(all-Z)-4,7,10,13,16,19-docosahexaen-1-ol from 3:1 to 1:3. All weight ratios above are also included for the pro-drugs of (all-Z) 5,8,11,14,17-eicosapentaen-1-ol and (all-Z)-4,7,10,13,16,19-docosahexaen-1-ol.

In an exemplary embodiment of the lipid composition according to the invention, at least 65% by weight of the omega-3 polyunsaturated alcohols is comprised of (all-Z)-5,8,11,14,17-eicosapentaen-1-ol and (all-Z)-4,7,10,13,16,19-docosahexaen-1-ol, in a weight ratio of (all-Z)-5,8,11,14,17-eicosapentaen-1-ol:(all-Z)-4,7,10,13,16,19-docosahexaen-1-ol from 3:1 to 1:3. In a more particular embodiment, at least 70% by weight of the omega-3 polyunsaturated alcohols is comprised of (all-Z)-5,8,11,14,17-eicosapentaen-1-ol and (all-Z)-4,7,10,13,16,19-docosahexaen-1-ol, in a weight ratio of (all-Z)-5,8,11,14,17-eicosapentaen-1-ol:(all-Z)-4,7,10,13,16,19-docosahexaen-1-ol from 1:2 to 2:1.

Further, in another exemplary embodiment of the invention, at least 70% by weight of the omega-3 polyunsaturated alcohols is comprised of (all-Z)-5,8,11,14,17-eicosapentaen-1-ol and (all-Z)-4,7,10,13,16,19-docosahexaen-1-ol, in a weight ratio of (all-Z) 5,8,11,14,17-eicosapentaen-1-ol:(all-Z)-4,7,10,13,16,19-docosahexaen-1-ol from about 0.0 to 1.5.

In another exemplary embodiment of the invention, the lipid composition is a pharmaceutical composition, a nutritional composition, or a dietary composition. These compositions may further comprise an effective amount of an acceptable antioxidant, e.g. tocopherol or mixtures of tocopherols, in an amount of up to 6 mg per gram, preferably 0.2 to 4 mg per gram, and most preferably 0.5 to 2 mg per gram. Moreover, all compositions according to the invention may be formulated for oral administration.

In an exemplary embodiment of the invention, the lipid composition is shaped in a form of a capsule, which could also be a microcapsule generating a powder or a sachet. The composition may also be present as a solid dosage form. The capsule may be flavoured. This embodiment also includes a capsule, wherein both the capsule and the encapsulated composition according to the invention is flavoured. By flavouring the capsule, it becomes more attractive to the user. For the therapeutic uses described herein the dosage administered will, of course, vary with the compound employed, the mode of administration, the treatment desired, and the disorder being treated or prevented.

The lipid composition may be formulated to provide a daily dosage of e.g. 0.1 g to 10 g; 0.5 g to 3 g; or 0.5 g to 1.5 g of the omega-3 polyunsaturated alcohols described herein, or prodrugs thereof. By a daily dosage is meant the dosage per 24 hours. The dosage administered will, of course, vary with the compound employed, the mode of administration, the treatment desired, and the disorder indicated. Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

A pharmaceutically active amount” relates to an amount that will lead to the desired pharmacological and/or therapeutic effects, i.e. an amount of the omega-3 polyunsaturated alcohols, or pro-drugs thereof, which is effective to achieve its intended purpose. While individual patient needs may vary, determination of optimal ranges for effective amounts of the omega-3 polyunsaturated alcohols, or pro-drugs thereof, are within the skill of the art. Generally, the dosage regimen for treating a condition with the compounds and/or compositions of this invention is selected in accordance with a variety of factors, including the type, age, weight, sex, diet, and medical condition of the patient.

By “a medicament” is meant a lipid composition according to the invention, in any form suitable to be used for a medical or non-medical purpose, e.g. in the form of a medicinal product, a pharmaceutical preparation or product, a dietary product, a food stuff or a food supplement, or a so called “lifestyle” supplement.

“Treatment” includes any therapeutic application that can benefit a human or a non-human mammal. Both human and veterinary treatments are within the scope of the present invention. Treatment may be for an existing condition or it may be prophylactic. An adult, a juvenile, an infant, a fetus, or a part of any of the aforesaid (e.g. an organ, tissue, cell, or nucleic acid molecule) may be treated.

The lipid composition may be used on their own but will generally be administered in the form of a pharmaceutical composition in which the omega-3 polyunsaturated alcohols, or prodrugs thereof, (the active ingredient) are in association with a pharmaceutically acceptable carrier, an excipient, a diluent, or a combination thereof. Moreover, acceptable carriers, excipients and diluents for therapeutic use are well-known in the pharmaceutical art, and can be selected with regard to the intended route of administration and standard pharmaceutical practice. Examples encompass binders, lubricants, suspending agents, coating agents, solubilising agents, preserving agents, wetting agents, emulsifiers, sweeteners, colourants, flavouring agents, odourants, buffers, suspending agents, stabilising agents, and/or salts.

A pharmaceutical composition according to the invention is preferably formulated for oral administration to a human or an animal. The pharmaceutical composition may also be formulated for administration through any other route where the active ingredients may be efficiently absorbed and utilized, e.g. intravenously, subcutaneously, intramuscularly, intranasally, rectally, vaginally, or topically.

The lipid composition may further comprise omega-3 polyunsaturated alcohols, or pro-drugs thereof, selected from the group consisting of (all-Z)-6,9,12,15,18-heneicosapentaen-1-ol, or a pro-drug thereof, (all-Z)-7, 10,13,16,19-docosapentaen-1-ol, or a pro-drug thereof, and (all-Z)-6,9,12,15-octadecatetraen-1-ol, or a pro-drug thereof.

In an exemplary embodiment of the invention, the lipid composition comprises at least pro-drugs of (all-Z)-5,8,11,14,17-eicosapentaen-1-ol chosen from a compound of formula (III),

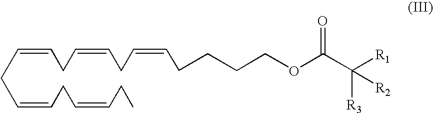

wherein,
R₁, R₂, and R₃ are chosen from:

a hydrogen atom,

a C₁-C₂₂ alkyl, and

a C₁-C₂₂ alkenyl with 1 to 6 double bonds in Z or E configuration, wherein the alkyl and alkenyl groups are optionally substituted, or a salt thereof.

Preferably, R is a C₁₂-C₂₂ polyunsaturated alkenyl with 2 to 6 methylene interrupted double bonds in Z configuration.

In an exemplary embodiment, the lipid composition comprises at least pro-drugs of (all-Z)-4,7,10,13,16,19-docosahexaen-1-ol chosen from a compound of formula (IV);

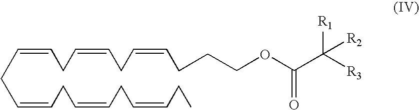

wherein
R₁, R₂, and R₃ are chosen from:

a hydrogen atom,

a C₁-C₂₂ alkyl, and

a C₁-C₂₂ alkenyl with 1 to 6 double bonds in Z or E configuration, wherein the alkyl and alkenyl groups are optionally substituted, or a salt thereof.

Preferably, R is a C₁₂-C₂₂ polyunsaturated alkenyl with 2 to 6 methylene interrupted double bonds in Z configuration. In an exemplary embodiment, a lipid composition according to the invention comprises at least a combination of the pro-drugs mentioned herein.

The present invention also relates to a lipid or pharmaceutical composition according to the invention for use as a medicament, a pharmaceutical, or for use in therapy.

Further, the invention relates to the use of a lipid composition, or a pharmaceutical composition, for the production of a medicament, a pharmaceutical and/or a food or nutritional supplement for:
the prevention and/or treatment of hypertriglyceridemia (HTG), dyslipidemia, hypertension and/or hypercholesteremia.
the prevention and/or treatment of elevated triglyceride levels, LDL cholesterol levels, and/or VLDL cholesterol levels.
the prevention and/or treatment of post-myocardial infarction (MI) or depression, heart failure, cardiac arrhythmias and/or atrial fibrillation.
the prevention and/or treatment of vascular diseases and/or atherosclerotic diseases.
the treatment and/or the prevention of obesity or an overweight condition.
the treatment and/or the prevention of reduction of body weight and fat mass and/or for preventing body weight gain.
the treatment and/or the prevention of an inflammatory disease or condition.

In an exemplary embodiment of the invention, the lipid composition, or pharmaceutical composition, according to the invention is used for treatment of hyperlipidemic conditions. In an exemplary embodiment, the present invention includes methods of blood lipid therapy in a subject comprising administering to the subject a pharmaceutically effective amount of a lipid composition according to the invention, wherein the subject has a baseline triglyceride level of 200 to 499 mg/dl, and wherein after administration to the subject the triglyceride level, and preferably a LDL cholesterol level, of the subject are reduced.

Moreover, the triglyceride level of a subject is generally as normal if less than 150 mg/dL, borderline to high if within about 150-199 mg/dL, high if within about 200-499 mg/dL and very high if 500 mg/dL or higher. The present invention may be used to reduce the triglyceride level of a “very high” down to a “high” or “high to borderline”.

Furthermore, the lipid composition comprising omega-3 polyunsaturated alcohols, or pro-drugs thereof as described herein, are useful for the treatment and prophylaxis of multiple risk factors known for cardiovascular diseases, such as hypertension, hypertriglyceridemia and high coagulation factor VII phospholipid complex activity. The omega-3 polyunsaturated alcohols, or pro-drugs thereof, acting as an lipid lowering or decreasing drug, may be used for the treatment of elevated blood lipids in humans.

In an exemplary embodiment of the invention, the invention provides for the use of omega-3 polyunsaturated alcohols, or pro-drugs thereof, for the manufacture of a medicament for lowering triglycerides in the blood of mammals and/or at the same time may increase HDL cholesterol levels in the serum of a human patients.

In an exemplary embodiment, a pharmaceutical composition for the treatment of elevated triglyceride levels comprises at least omega-3 polyunsaturated alcohols in a concentration of at least 80% by weight as compared to the total lipid content of the composition, and wherein at least 70% of the omega-3 polyunsaturated alcohols is comprised of a combination of (all-Z)-5,8,11,14,17-eicosapentaen-1-ol and (all-Z)-4,7,10,13,16,19-docosahexaen-1-ol in a weight ratio of (all-Z)-5,8,11,14,17-eicosapentaen-1-ol:(all-Z)-4,7,10,13,16,19-docosahexaen-1-ol from 0.5:3 to 3:0.5.

In another exemplary embodiment, a pharmaceutical composition according to the invention may also provide an increased effect on inflammatory diseases, including chronic inflammatory diseases characterized by leukocyte accumulation and leukocyte-mediated tissue injury, neural development and visual functions. In an exemplary embodiment, the present invention also provides for the use of a lipid composition according to the invention for the manufacture of a medicament or pharmaceutical for the treatment and/or the prevention of atherosclerosis, psoriasis, multiple sclerosis and/or rheumatoid arthritis.

A lipid composition according to the invention may also be used for the prevention and/or treatment of amyloidos-related diseases. Amyloidos-related conditions or diseases associated with deposition of amyloid, preferably as a consequence of fibril or plaque formation, includes Alzheimer's disease or dementia, Parkinson's disease, amyotropic lateral sclerosis, the spongiform encephalopathies, such as Creutzfeld-jacob disease, cystic fibrosis, primary or secondary renal amyloidoses, IgA nephropathy, and amyloid depostion in arteries, myocardium and neutral tissue. These diseases can be sporadic, inherited or even related to infections such as TBC or HIV, and are often manifested only late in life even if inherited forms may appear much earlier. Particular protein or aggregates of those proteins are thought to be the direct origin of the pathological conditions associated with these diseases. The treatment of a amyloidos-related disease can be made either acutely or chronically.

The polyunsaturated alcohols, or prodrugs, according to the invention may also be used for the treatment due to reduction of amyloid aggregates, prevention of misfolding of proteins that may lead to formation of so called fibrils or plaque, treatment due to decreasing of the production of precursor protein such as Aβ-protein (amyloid beta protein), and prevention and/or treatment due to inhibiting or slow down the formation of protein fibrils, aggregates, or plaque. Prevention of fibril accumulation, or formation, by administering compounds of formula (I), as hereinbefore defined, is also included herein. In one embodiment, the novel lipid compostions are used for the treatment of TBC (tuberculosis) or HIV (human immunodeficiency virus). Further, a lipid composition according to the invention may be administered to patients with symptoms of atherosclerosis of arteries supplying the brain, for instance a stroke or transient ischaemic attack, in order to reduce the risk of a further, possible fatal, attack.

The present invention relates to the use of an lipid composition comprising omega-3 polyunsaturated alcohols, or pro-drugs thereof, according to the invention for the manufacture of a medicament or pharmaceutical for the treatment and/or the prevention of at least one of atherosclerosis or IgA Nephropathy, prophylaxis of multiple risk factors for cardiovascular diseases, heart failure, atrial fibrillation and/or a post-myocardial infarct, stroke, treatment of TBC or HIV, and treatment of HTG in HIV patients.

Moreover, nonalcoholic fatty liver disease is a common condition associated with metabolic syndrome. More specifically, fatty liver is primary associated with hyperinsulinemia and insulin-resistance. In one embodiment of the invention a lipid composition comprising omega-3 polyunsaturated alcohols, or pro-drugs thereof, may act as an insulin-sensitizing agent and reduce liver steatosis. Moreover, fatty liver disease occurs in two major forms—alcoholic and nonalcoholic. Both terms are marked by accumulation of fat in the liver with variable amounts of liver injury, inflammation, and fibrosis. The spectrum of fatty liver disease ranges from simple steatosis (considered benign and non-progressive), to steatohepatitis (fatty liver with liver cell injury and inflammation), to progressive hepatic fibrosis and cirrhosis. All these conditions are included in the prevention and/or treatment with at least omega-3 polyunsaturated alcohols, or pro-drugs thereof, according to the invention.

The invention also relates to methods for the prevention and/or treatment of all conditions and diseases mentioned above, comprising administering to a patient, preferably a mammal in need thereof, a pharmaceutically active amount of a lipid composition according to the invention. An exemplary embodiment relates to a method for reducing abnormal triglyceride levels in a patient, preferably patients having triglyceride levels of about 200 to about 499 mg/dl before treatment, wherein a therapeutically effective amount of the lipid composition according to the invention is administered to a human or an animal.

Furthermore, the present invention encompasses a method for manufacturing lipid compositions according to the invention. Preferably, said lipid composition is prepared from a vegetable, a microbial and/or an animal source, more preferably from a marine oil, and most preferably from a fish oil or a krill oil.

One advantage of preparing omega-3 polyunsaturated alcohols, or pro-drugs thereof, according to the invention, is that it is possible to start with a mixed fatty acid composition, comprising omega-3 fatty acids or esters, known in the art, and then to carry out a reduction step, by reduction of the acids or esters, to their respective alcohols.

In an exemplary embodiment, the lipid composition according to the invention is prepared directly from a pre-concentrated mixed-fatty acid composition comprising at least 70% of weight of omega-3 fatty acid esters, comprising esters of at least the omega-3 C 20:5 and C 22:6 acids, wherein the esters of the omega-3 C 20:5 and C 22:6 acids are reduced to polyunsaturated alcohols by using a reagent that transfers a hydride to the carbonyl compound. Preferably, the reagent is chosen from lithium aluminium hydrides, such as LiAlH₄, LiAlH₂(OCH₂CH₂OCH₃), or LiAlH[OC(CH₃)₃]₃, and boron hydrides such as LiBH₄, or Ca(BH₄)₂.

Preferred compounds and compositions, according to the invention are divided into the following categories A-B;

Category A: Lipid compounds [pro-drugs derived from EPA-, DHA-, and ALA-alcohols]

### Pivaloate Esters

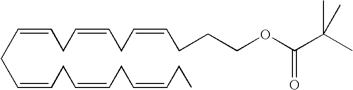

(all-Z)-4,7,10,13,16,19-docosahexaen-1-yl pivaloate

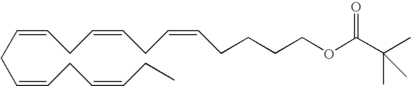

(all-Z)-5,8,11,14,17-eicosapentaen-1-yl pivaloate

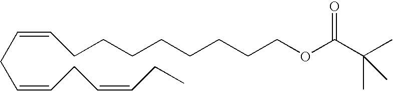

(all-Z)-9,12,15-octadecatrien-1-yl pivaloate

### Hemisuccinate Esters

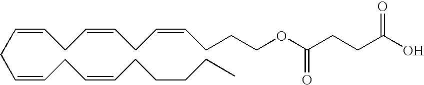

(all-Z)-4,7,10,13,16,19-docosahexaen-1-yl hemisuccinate

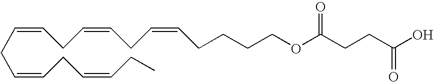

(all-Z)-5,8,11,14,17-eicosapentaen-1-yl hemisuccinate

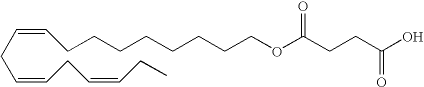

(all-Z)-9,12,15-octadecatrien-1-yl hemisuccinate

### Salt Forms of Hemisuccinate Esters

The different salts are described by using (all-Z)-4,7,10,13,16,19-docosahexaen-1-yl hemisuccinate as a non limiting example.

a)

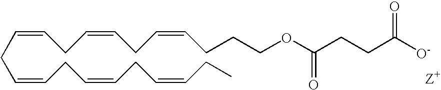

wherein Z⁺ is selected from the group consisting of Li⁺, Na⁺, K⁺, NH₄⁺,

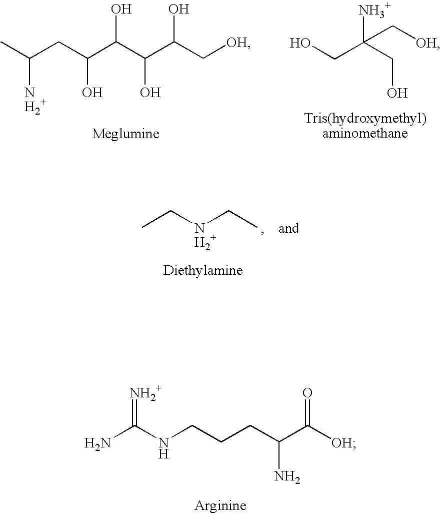

b)

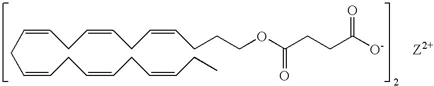

wherein Z² is selected from the group consisting of Mg²⁺, Ca²⁺,

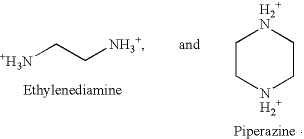

### c) Further Optional Salts

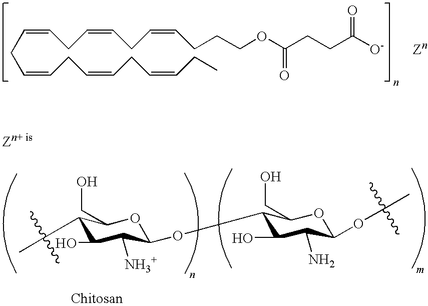

esters with polyunsaturated fatty acids

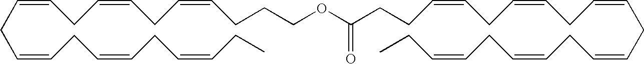

[(all-Z)-4,7,10,13,16,19-docosahexaen-1-yl] (all-Z)-3,6,9,12,15,18-docosahexaenoate

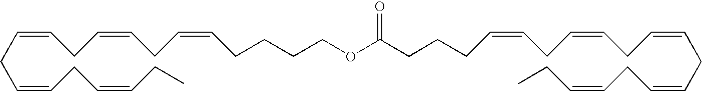

[(all-Z)-5,8,11,14,17-eicosapentaen-1-yl] (all-Z)-4,7,10,13,16-eicosapentaenoate

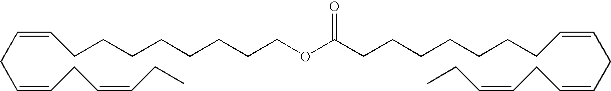

[(a Z)-9,12,15-octadecatrien-1-yl] (all-Z)-8,11,14-octadecatrienoate

Category B: Lipid Compositions

Lipid Composition Comprising Omega-3 Polyunsaturated Alcohols

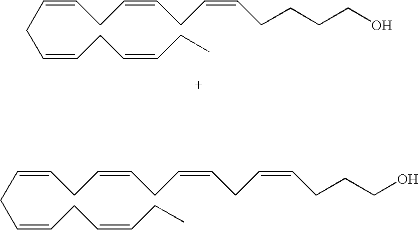

Lipid Composition Comprising Pro-Drugs of the Alcohols in the Form of Omega-3 Acetate Esters

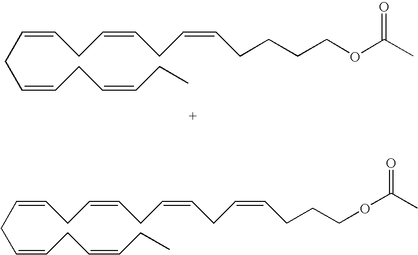

Lipid Composition Comprising Pro-Drugs of the Alcohols in the Form of Pivaloate Esters

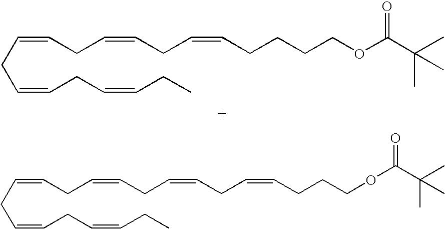

Lipid Composition Comprising Pro-Drugs of the Alcohols in Form of Hemisuccinate Esters

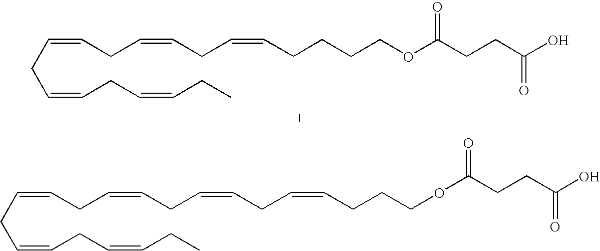

Lipid Composition Comprising Pro-Drugs of the Alcohols in the Form of Salts of Hemisuccinate Esters

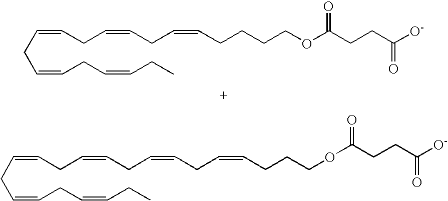

Lipid Composition Comprising Pro-Drugs of the Alcohols in the Form of Esters with Polyunsaturated Fatty Acids

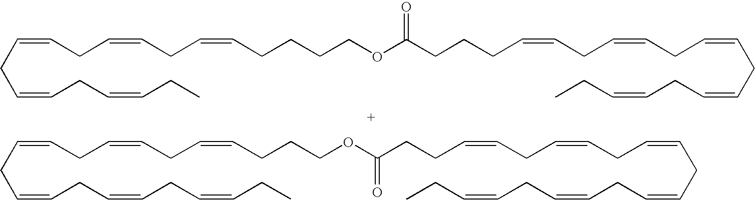

### Methods

### Methods for Preparing Omega-3 Polyunsaturated Alcohols, or Pro-Drugs Thereof.

A selection of methods for preparing mixed compositions comprising at least EPA and DHA, either in the form of esters, triglycerides, or free fatty acids, are presented below. All of these compositions, or intermediate compositions, can be reduced to their corresponding alcohols, which are included within the scope of the present invention.

Initially, the oil raw material, which may be a marine oil, is esterified to produce fatty acid ethyl esters. Subsequent processing steps include short path distillation and urea fractionation to increase the concentration of EPA and DHA. Fractionation of the fatty acid esters are carried out at conditions sufficiently mild to avoid disintegration of the products.

Short path distillation fractionates according to fatty acid molecular weight, and this processing step removes the major part of the esters having chain length below C20. The short path distillation is preferentially carried out in two distillation stages.

Urea forms complexes with fatty acids and fatty acids esters according to their degree of unsaturation. Urea is dissolved in a solvent, usually ethanol, and upon addition of the fatty acid esters, complexes of urea and the saturated and less unsaturated esters are formed. After removing the urea precipitate, the solvent is removed by evaporation, and the esters thus isolated are purified by washing with water. The product fraction contains high concentrations of EPA and DHA.

The product fraction from the urea complexation step may be further purified to remove unwanted components, such as oxidation by-products, by the treatment with bleaching earth or other polar adsorbents.

Other methods for production of fatty acid mixtures enriched in EPA and DHA are described in WO 95/24459, WO 2000/049117 and WO 2004/043894. In these processes the concentration of EPA and/or DHA is increased by a combination of lipase catalyzed esterification reactions and short-path distillations.

WO 95/24459 describes ethanolysis of fish oil triglycerides catalysed by a Pseudomonas lipase highly selective towards short-chain fatty acids. In this process a major part of short-chain fatty acids are converted to ethyl esters. In the following short-path distillation, these ethyl esters are distilled off leaving a glyceride fraction enriched in EPA and DHA.

WO 2000/049117 describes glycerolysis of a fish oil fatty acid mixture on ethyl ester or free fatty acid forms catalysed by a Rhizomucor miehei lipase highly selective towards short-chain fatty acids. In contrast to the Pseudomonas lipase above, the Rhizomucor miehei lipase has much higher selectivity toward EPA relative to DHA. By choosing the reaction conditions, both EPA and shorter fatty acids can be converted to glycerides. In a subsequent short-path distillation, a DHA-rich fraction ofs ethyl ester or free fatty acid forms is distilled off leaving the less volatile glyceride fraction as residue.

WO 2004/043894 describes ethanolysis of a fish oil fatty acid mixture of free fatty acid forms catalysed by the same Rhizomucor miehei as above. In this reaction a major part of the fatty acids C20 and shorter are converted to ethyl esters. Since ethyl esters are more volatile than free fatty acids, the subsequent short-path distillation will produce a residue enriched in DHA in free fatty acid form.

A. Ganga et al, JAOCS, Vol. 75, no. 6. 1998, describes a procedure to obtain 90% eicosapentaeinoic acid and docosahexaenoic acid concentrates from sardine oil by a two step winterisation of the oil, followed by saponification and selective precipitation of saturated and less unsaturated free fatty acids by an ethanolic solution of urea.

### Method I

Reduction of Mixtures of Omega-3 Polyunsaturated Ethyl Esters to their Corresponding Alcohols

Concentrates of polyunsaturated esters can be reduced to their corresponding alcohols by using a reagent that transfers a hydride to the carbonyl compound. Non-limiting examples of such reducing agents are: lithium aluminium hydrides, such as LiAlH₄, LiAlH₂(OCH₂CH₂OCH₃), LiAlH[OC(CH₃)₃]₃ and boron hydrides, such as LiBH₄ and Ca(BH₄)₂.

### EXAMPLES

The invention will now be described in more detail by the following examples, which are not to be constructed as limiting the invention.

In some of the examples a lipid mixture containing 90% omega-3 PUFAs as ethylesters was used as starting material. The mixture contained approximately 85% w/w of ethyl (all-Z)-5,8,11,14,17-eicosapentaenoate and ethyl (all-Z)-4,7,10,13,16,19-docosahexaenoate in a ratio of 1.2 w/w. For simplicity this mixture is called K85EE.

In some of the examples a lipid mixture containing approximately 55% omega-3 PUFAs as ethylesters was used as staring material. The mixture contained approximately 50% w/w of ethyl (all-Z)-5,8,11,14,17-eicosapentaenoate and ethyl (all-Z)-4,7,10,13,16,19-docosahexaenoate. For simplicity this mixture is called K50EE

Other PUFA ethylester mixtures can be used as staring material.

### Example 1

### Reduction of K85EE to K85 Alcohol

The structures were verified by NMR and by Mass Spectrometry (MS). The NMR spectra were recorded in CDCl₃. J values are given in Hz.

A suspension of LiAlH₄ (0.11 g, 3.0 mmol) in dry THF (10 mL) under inert atmosphere was given 0° C. and K85EE (1.00 g, 2.9 mmol) in dry THF (15 mL) was added dropwise. The mixture was stirred at 0° C. for 15 minutes, added 10% NH₄Cl (20 mL) and filtrated through a short pad of celite. The pad was washed with water (20 mL) and heptane (20 mL) and the layers were separated. The aqueous phase was extracted with heptane (20 mL) and the combined organic layer was washed with brine (20 mL) and dried (MgSO₄). This afforded 0.75 g (84%) of the title compound as a 1:1 mixture of (all-Z) 5,8,11,14,17-eicosapentaen-1-ol and (all-Z)-4,7,10,13,16,19-docosahexaen-1-o\ as an oil.

¹H-NMR (200 MHz, CDCl₃): δ 0.94 (t, 3H), 1.24-1.60 (m, 6H), 1.80 (m, 1H), 1.98-2.17 (m, 4H), 2.76-2.90 (m, 9H), 3.60 (t, 4H), 5.27-5.48 (m, 11H). ¹³C-NMR (50 MHz, CDCl₃): δ 14.03, 14.18, 20.47, 22.61, 23.50, 25.46, 25.56, 25.68, 26.87, 28.94, 31.80, 32.24, 32.39, 62.29, 62.66, 126.94, 127.78, 127.91, 127.97, 128.00, 128.05, 128.12, 128.17, 128.22, 128.30, 128.36, 128.47, 129.36, 129.82, 131.93. MS (ESI): 311/337 [M+Na⁺]⁺.

### Example 2

### Reduction of K-50EE, (Preparation of K-50-ol)

K-50EE (100 g) in 450 mL dry THF was added drop wise to a stirred suspension of LiAlH₄ (11.56 g, 0.304 mol) in 500 mL dry THF held at 0° C. The mixture was stirred at 0° C. under inert atmosphere for 2.5 h, added 10% NH₄Cl (200 mL) and filtrated through a short pad of celite. The pad was washed with water (250 mL) and heptane (250 mL) and the layers were separated. The aqueous phase was extracted with heptane (500 mL) and the combined organic layer was washed with brine (200 mL) and dried (Na₂SO₄). This afforded 77.82 g of the title compound as a mixture of EPA-OH and DHA-OH (and other unidentified compounds) as a yellow oil. ¹H-NMR (200 MHz, CDCl₃): δ 0.95 (t, 3H, J=7.5 Hz), 1.23-1.39 (m, 15.6H), 1.41-1.43 (m, 2.6H), 1.50-1.65 (m, 3.4H), 1.98-2.15 (m, 5.5H), 2.76-2.85 (m, 8.4H), 3.58-3.66 (m, 3H), 5.31-5.44 (m, 10.9H); MS (electrospray): 118.1, 128.9, 311.2 [EPA-OHh+Na]⁺, 337.2 [DHA-OH+Na]⁺

### Method II

### Reduction to Alcohols at an Early Stage in a Purification Process

Instead of producing the concentrates of the polyunsaturated esters prior to reduction (see method I) it is a possibility to do the reduction step at an earlier stage in the purification process. A reduction of, for instance, a crude fish oil will give a mixture of lipid alcohols. This lipid alcohol mixture will contain structurally different alcohols derived from both saturated lipids and polyunsaturated lipids and with different chain length. These alcohol mixtures can be purified by purification technologies well-known in the art.

### Method III

Variations of method II described above might include trans-esterification of for instance a crude fish oil to a mixture of esters. This ester mixture can be distilled prior to the reduction procedure. After reduction, the alcohol mixture can be purified according to methods well-known in the art.

### Method IV

Preparation of Pro-Drugs of omega-3 Polyunsaturated Alcohols

General methods to synthesize esters from lipid alcohols include reactions of alcohols with an acid chloride or other activated carboxylic acid derivatives. Preparative procedures often use pyridine as a catalyst when reacting the alcohol with an acid chloride. 4-dimethyl-aminopyridine (DMAP) is an alternative catalyst for this reaction. A Fisher esterification procedure, where a lipid alcohol is reacted with a carboxylic acid in the presence of an acid-catalyst can also be used to prepare pro-drugs omega-3 polyunsaturated alcohols.

Scheme (A) illustrates an example for preparation of pro-drugs of omega-3 polyunsaturated alcohols. A lipid composition comprising omega-3 polyunsaturated alcohols, primary (all-Z)-5,8,11,14,17 eicosapentaen-1-ol and (all-Z)-4,7,10,13,16,19-docosahexaen-1-ol, is reacted with acetyl chloride in the presence of pyridine to produce one of the pro-drugs according to the invention.

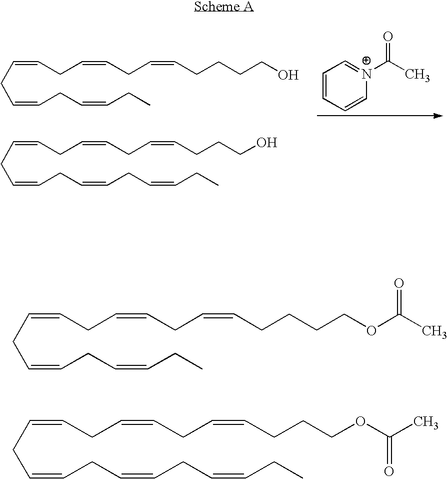

Omega-3 polyunsaturated alcohols, or pro-drugs thereof, can be manufactured from raw materials other than marine oils, according to the same methods and principles available for the production of omega-3 concentrates with EPA and DHA, such as algae oils and oils from genetically modified plants.

### Examples

The invention will now be described in more detail by the following examples, which are not to be constructed as limiting the invention.

### Example 2

### Synthesis of the acetate of K85 Alcohol (Scheme A)

A solution of K85 alcohol (example 1, 186.1 g) in THF (800 ml) was cooled to 0° C. under N₂. Pyridine (2.0 ml, 25 mmol) was added. The resulting mixture was stirred for 15 minutes and acetyl chloride (48.3 ml, 680 mmol) was added. The mixture was stirred at room temperature for 20 hrs. Heptane (1 L) was added and the resulting mixture was washed with sat. NaHCO₃ (300 ml) and water (800 ml), dried (Na₂SO₄) and evaporated in vacuo. The crude product was dissolved in heptane (500 ml) and filtered through a short pad of silica. Yield: 206.7 g as a 1:1 mixture of (all-Z)-5,8,11,14,17-eicosapentaen-1-ol acetate ester and (all-Z)-4,7,10,13,16,19-docosahexaen-1-ol acetate ester as a oil.

¹H NMR (200 MHz, CDCl₃): δ 0.93 (t, J=7.5 Hz, 3H), 1.22-1.42 (m, 3.2H), 1.56-1.69 (m, 2.3H), 1.98 (s, 4H), 1.98-2.31 (m, 3.3H), 2.74-3.11 (m, 8.8H), 4.02 (t, J=6.5 Hz, 2.2H), 5.04-5.41 (m, 10.8H); ¹³C-NMR (50 MHz, CDCl₃): δ 13.9, 14.1, 20.4, 20.7, 22.5, 23.4, 25.35, 25.39, 25.4, 25.8, 26.6, 26.9, 27.0, 28.0, 28.3, 28.8, 29.1, 31.3, 31.7, 63.6, 64.2, 126.8, 127.7, 127.85, 127.90, 127.96, 128.01, 128.1, 128.3, 128.5, 128.6, 129.4, 129.7, 129.9, 131, 7, 136.5, 170.7, 170.8; MS (ESI); 353/379 [M+Na⁺]⁺

### Example 3

### (all-Z)-5,8,11,14,17-eicosapentaen-1-ol pivaloate ester

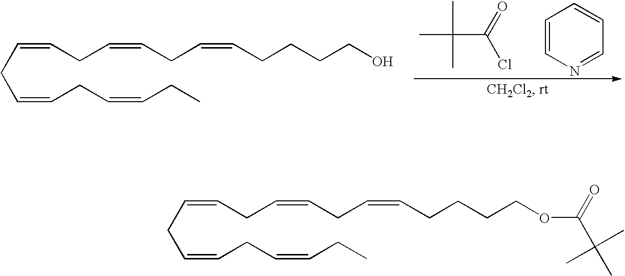

Pivaloyl chloride (225 μl, 1.83 mmol) was added to a mixture of (all-Z)-5,8,11,14,17-eicosapentaen-1-ol (501 mg, 1.74 mmol) and pyridine (0.14 ml, 1.73 mmol) in dry CH₂Cl₂ (3 ml) at room temperature under nitrogen, and the resulting mixture was stirred for 18 h. Diethyl ether (50 ml) was added and the resulting mixture was washed with water (20 ml) and brine (20 ml), dried (Na₂SO₄) and evaporated in vacuo. The residue was purified by flash chromatography (SiO₂, heptane/ethyl acetate 100:1). Yield: 440 mg (68%)

¹H NMR (200 MHz, CDCl₃) δ 0.95 (t, J=7.5 Hz, 3H), 1.17 (s, 9H), 1.34-1.48 (m, 2H), 1.54-1.70 (m, 2H), 1.98-2.13 (m, 4H), 2.77-2.85 (m, 8H), 4.04 (t, J=6.4 Hz, 2H), 5.23-5.43 (m, 10H); MS (ESI); 395 [M+Na⁺]⁺

### Example 4

### (all-Z)-5,8,11,14,17-eicosapentaen-1-1/hemisuccinate

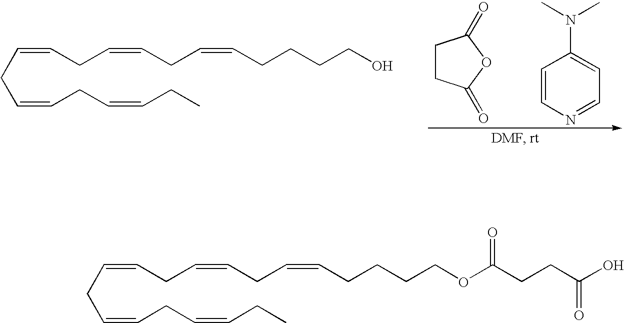

A mixture of (all-Z)-5,8,11,14,17-eicosapentaen-1-ol (501 mg, 1.74 mmol), succinic acid anhydride (183 mg, 1.83 mmol) and DMAP (212 mg, 1.74 mmol) in dry DMF (3 ml) was stirred at room temperature under nitrogen for 19 hrs. Diethyl ether (50 ml) was added and the resulting mixture was washed with 1M HCl (20 ml) and brine (20 ml), dried (Na₂SO₄) and evaporated in vacuo. The residue was purified by flash chromatography (SiO₂, heptane/ethyl acetate 95:5-1:1). Yield: 232 mg (34%)

¹H NMR (200 MHz, CDCl₃) δ 0.95 (t, J=7.5 Hz, 3H), 1.36-1.47 (m, 2H), 1.56-1.70 (m, 2H), 1.98-2.12 (m, 4H), 2.55-2.71 (m, 4H), 2.76-2.89 (m, 8H), 4.08 (t, J=6.5 Hz, 2H), 5.22-5.43 (m, 10H); MS (ESI); 387 [M-H⁺]⁻.

### Method V

### Preparation of Esters of Omega-3 Polyunsaturated Alcohols and Acids

A general method for the preparation of the esters with polyunsaturated fatty acids involves reacting one equivalent of the polyunsaturated fatty acid with one equivalent of the polyunsaturated alcohol in the precence of EDC (1-Ethyl-3-[3-dimethylaminopropyl]carbodiimide hydrochloride), or another activator for carboxylic acids, and a base (like triethylamine or diisopropylethylamine) in an appropriate solvent.

An example is shown in Scheme B.

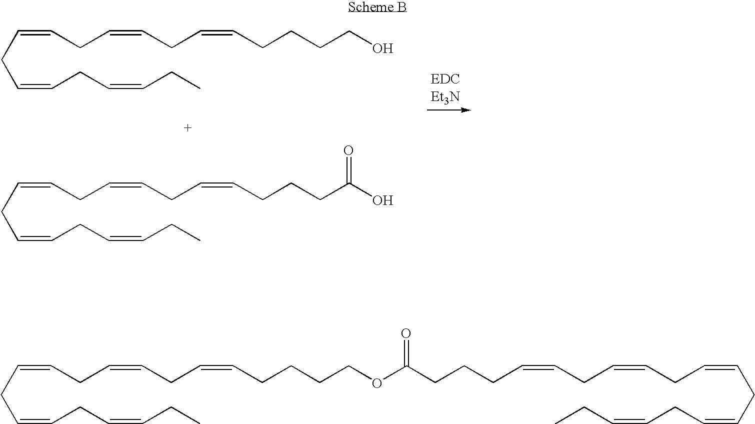

### Effect Studies

### Test Example 1

### Demonstration of Effects on Lipid Metabolism In Vivo

The invented compositions were tested in an animal model as described below.

Mice

Female heterozygous APOE*3Leiden mice was used, and housed during the experiment in macrolon cages (three or four mice per cage), in clean-conventional animal rooms (relative humidity 50-60%, temperature ˜21° C., light cycle 7 am to 7 pm). Individual animals were marked by ear punch-holes. Mice were supplied with food and acidified tap water ad libitum.

Diets

The mice received a semi-synthetic modified Western-type diet (WTD) as described by Nishina et al (J Lipid Res 1990; 31: 859), containing cholesterol (0.25% w/w, final concentration) and 15% cacaobutter.

Drug Administration

All test compounds were administered orally as admix to the Western-type diet. The lyophilized diet chunks were stored in vacuum bags in the dark in an alarm-secured −20° C. room. The diets on the cages of the mice were changed twice a week.

Study Design

APOE*3Leiden mice were put on a semi-synthetic Western-type diet (WTD, 15% cocoa butter, 40% sucrose and 0.25% cholesterol; all w/w). After a 4 weeks run-in period low-responder mice were removed from the study and the remaining mice were sub-divided into five groups of 10 mice each, matched for plasma cholesterol, triglycerides, free fatty acids and age (t=0)

The five groups were treated with:

Group 1: WTD without addition, control

Group 2: WTD plus acetates derived from K85ol

Group 3: WTD plus alcohols (K85ol) derived from K85 ethyl ester

Group 4: WTD plus Fenofibrate

After 3 weeks of treatment (t=3 weeks) blood samples were taken after 4 hour-fast period and plasma total cholesterol (TC), total triglycerides (TG) were measured. [Delta values are defined as: plasma levels at t=0 minus plasma levels at t=3] The results are shown in table 1 and 2. As evident from these results, it was shown that all the inventive compounds had lipid lowering effects.

Results:

TABLE 1
Delta cholesterol plasma levels (delta
TC) after treatment period of 3 weeks

| | | | Mean | Std |
|---|---|---|---|---|
| Substance | Dose | Number | (delta TC) | Deviation |
| Group 1 | Control | 10 | 1.02 | 2.276 |
| Group 2 | 565 | 10 | 6.32 | 3.077 |
| | mg/kg | | | |
| | bw/d | | | |
| Group 3 | 497 | 10 | 4.76 | 2.632 |
| | mg/kg | | | |
| | bw/d | | | |
| Group 4 | 1 | 10 | 4.71 | 2.324 |
| | mg/kg | | | |
| | bw/day | | | |

TABLE 2
Delta triglyceride plasma levels (delta
TG) after treatment period of 3 weeks

| | | | Mean | Std |
|---|---|---|---|---|
| Substance | Dose | Number | (delta TG) | Deviation |
| Group 1 | Control | 10 | 0.77 | 0.889 |
| Group 2 | 565 | 10 | 1.30 | 0.664 |
| | mg/kg | | | |
| | bw/d | | | |
| Group 3 | 497 | 10 | 1.15 | 0.761 |
| | mg/kg | | | |
| | bw/d | | | |
| Group 4 | 1 | 10 | 1.16 | 0.521 |
| | mg/kg | | | |
| | bw/day | | | |

Formulations and Compositions

Processes for the fractionation of fatty acids or fatty acid alkyl esters from marine oils may be carried out separately or combined in order to produce mixed-fatty acid compositions with concentrations of EPA and DHA varying over a wide range, and the samples available commercially reflect this. The concentrations of EPA and DHA depend on the concentration in the starting material and the fractionation process used, as well as the process yield. Processes used commercially include short path distillation, supercritical fluid fractionation, urea complexation, preparative chromatography and extrography.

Fractionation of fatty acids from marine oils by short path distillation or supercritical fluid fractionation commonly produces long-chain polyunsaturated omega-3 oils with a concentration of EPA+DHA of 50-60% by weight, typically containing 30-40% EPA and 20-30% DHA. Commercial examples of such mixed-fatty acid compositions are EPAX5500TG and EPAX6000FA (EPAX A.S.), K50EE (Pronova Biocare A.S.), Incromega E3322 and Incromega TG3322 (Croda), and MEG-3 Concentrate 30/20 EE and MEG-3 Concentrate 40/20 TG (Ocean Nutrition Canada). These compositions may be in the form of alcohols, or pro-drugs thereof, according to the invention (instead of in the form of esters, triglyceride, free fatty acids).

Particular fractionation may be carried out in order to produce high purity long-chain polyunsaturated omega-3 oils, typically EPA+DHA >75%. Commercial examples of such mixed-fatty acid compositions are K70EE, K80EE, K85EE, K85TG, and AGP103 (Pronova BioPharma Norge AS), which compositions may be in the form of alcohols, or pro-drugs thereof, according to the invention (instead of in the form of esters, triglyceride, free fatty acids). Another commercial example is a the pharmaceutical product EPAdel (high concentrated EPA lipid product).

Moreover, fractionation of fatty acids or ethyl esters may be carried out in such a way as to manufacture long-chain polyunsaturated omega-3 oils which are selectively enriched in EPA. Commercial examples of such mixed-fatty acid compositions are EPAX4510TG and EPAX7010EE (EPAX A.S.), Incromega EPA500TG and Incromega E7010 SR (Croda), and MEG-3 60/03TG and MEG-3 50/20EE (Ocean Nutrition Canada), which compositions may be in the form of alcohols, or pro-drugs thereof, according to the invention (instead of in the form of esters, triglyceride, free fatty acids).

Additionally, fractionation of fatty acids or fatty acid ethyl esters may be carried out in such a way as to manufacture long-chain omega-3 oils which are selectively enriched in DHA. Commercial examples of such mixed-fatty acid compositions are EPAX2050TG (EPAX A.S.), Incromega DHA500TG and Incromega 700E SR (Croda), and MEG-3 20/50TG and MEG-3 05/55EE (Ocean Nutrition Canada), which compositions may also be in the form of alcohols, or prodrugs thereof, according to the invention (instead of in the form of esters, triglyceride, free fatty acids).

Thus, all alcohols and pro-drugs of the commercial examples mentioned herein are included as embodiments of the present invention.

The most preferred form of a lipid composition according to the invention is the omega-3 alcohols or acetates of the Omacor® omega-3 ethyl ester, i.e. K85EE (Pronova Biocare A.S., Lysaker, Norway), and preferably comprises the lipid composition possessing the following characteristics (per dosage form (1000 mg)):

| | Minimum | | Maximum |
|---|---|---|---|
| K85 alcohol | value | | value |
| (all-Z)-5,8,11,14,17 eicosapentaen-1-ol | 430 | mg/g | 495 mg/g |
| (all-Z)-4,7,10,13,16,19-docosahexaen-1-ol | 347 | mg/g | 403 mg/g |
| (all-Z)-5,8,11,14,17 eicosapentaen-1-ol and | 800 | mg/g | 880 mg/g |
| (all-Z)-4,7,10,13,16,19-docosahexaen-1-ol | | | |
| Total omega-3 polyunsaturated alcohols | 90% | (w/w) | |

1. A lipid composition comprising omega-3 polyunsaturated alcohols, wherein the omega-3 polyunsaturated alcohols comprise (all-Z)-5,8,11,14,17-eicosapentaen-1-ol and (all-Z)-4,7,10,13,16,19-docosahexaen-l-ol.

2. A lipid composition according to claim 1, wherein the omega-3 polyunsaturated alcohols are present in a concentration of least 30% by weight of the total lipid content of the composition.

3. A lipid composition according to claim 1, wherein the omega-3 polyunsaturated alcohols are present in a concentration of least 50% by weight of the total lipid content of the composition.

4. A lipid composition according to claim 1, wherein the omega-3 polyunsaturated alcohols are present in a concentration of least 70% by weight of the total lipid content of the composition.

5. A lipid composition according to claim 1, wherein the omega-3 polyunsaturated alcohols are present in a concentration of least 80% by weight of the total lipid content of the composition.

6. A lipid composition according to claim 1, wherein (all-Z) 5,8,11,14,17-eicosapentaen-1-ol and (all-Z)-4,7,10,13,16,19-docosahexaen-1-ol comprise 20% by weight of omega-3 polyunsaturated alcohols in the composition.

7. A lipid composition according to claim 6, wherein (all-Z) 5,8,11,14,17-eicosapentaen-1-ol and (all-Z)-4,7,10,13,16,19-docosahexaen-1-ol comprise 40% by weight of omega-3 polyunsaturated alcohols in the composition.

8. A lipid composition according to claim 7, wherein (all-Z) 5,8,11,14,17-eicosapentaen-l-ol and (all-Z)-4,7,10,13,16,19-docosahexaen-l-ol comprise 70% by weight of omega-3 polyunsaturated alcohols in the composition.

9. A lipid composition according to claim 8, wherein (all-Z) 5,8,11,14,17-eicosapentaen-l-ol and (all-Z)-4,7,10,13,16,19-docosahexaen-l-ol comprise 80% by weight of omega-3 polyunsaturated alcohols in the composition.

10. A lipid composition according to claim 1, wherein (all-Z) 5,8,11,14,17-eicosapentaen-1-ol comprises 5% to 95% by weight of the total lipid content in the composition.

11. A lipid composition according to claim 10, wherein (all-Z) 5,8,11,14,17-eicosapentaen-1-ol comprises 40% to 55% by weight of the total lipid content in the composition.

12. A lipid composition according to claim 1, wherein (all-Z)-4,7,10,13,16,19-docosahexaen-1-ol comprises 5% to 95% by weight of the total lipid content in the composition.

13. A lipid composition according to claim 12, wherein (all-Z)-4,7,10,13,16,19-docosahexaen-l-ol comprises 30% to 60% by weight of the total lipid content in the composition.

14. A lipid composition according to claim 1, wherein the omega-3 polyunsaturated alcohols comprise (all-Z) 5,8,11,14,17-eicosapentaen-l-ol and (all-Z)-4,7,10,13,16,19-docosahexaen-1-ol in a weight ratio of (all-Z) 5,8,11,14,17-eicosapentaen-l-ol:(all-Z)-4,7,10,13,16,19-docosahexaen-l-ol from 99:1 to 1:99.

15. A lipid composition according to claim 14, wherein the omega-3 polyunsaturated alcohols, comprise (all-Z)-5,8,11,14,17-eicosapentaen-1-ol and (all-Z)-4,7,10,13,16,19-docosahexaen-l-ol in a weight ratio of (all-Z) 5,8,11,14,17-eicosapentaen-l-ol:(all-Z)-4,7,10,13,16,19-docosahexaen-l-ol from 10:1 to 1:10.

16. A lipid composition according to claim 15, wherein the omega-3 polyunsaturated alcohols comprise (all-Z) 5,8,11,14,17-eicosapentaen-l-ol and (all-Z)-4,7,10,13,16,19-docosahexaen-l-ol in a weight ratio of (all-Z)-5,8,11,14,17-eicosapentaen-l-ol:(all-Z)-4,7,10,13,16,19-docosahexaen-1-ol from 5:1 to 1:5.

17. A lipid composition according to claim 16, wherein the omega-3 polyunsaturated alcohols comprise (all-Z) 5,8,11,14,17-eicosapentaen-1-ol and (all-Z)-4,7,10,13,16,19-docosahexaen-l-ol in weight a ratio of (all-Z)-5,8,11,14,17-eicosapentaen-1-ol:(all-Z)-4,7,10,13,16,19-docosahexaen-1-ol from 1:2 to 2:1.

18. A lipid composition according to claim 16, wherein the omega-3 polyunsaturated alcohols comprise (all-Z) 5,8,11,14,17-eicosapentaen-1-ol and (all-Z)-4,7,10,13,16,19-docosahexaen-1-ol in a weight ratio of (all-Z)-5,8,11,14,17-eicosapentaen-1-ol:(all-Z)-4,7,10,13,16,19-docosahexaen-1-ol of about 1.2.

19. A lipid composition according to claim 16, wherein the omega-3 polyunsaturated alcohols comprise (all-Z) 5,8,11,14,17-eicosapentaen-1-ol and (all-Z)-4,7,10,13,16,19-docosahexaen-1-ol in a weight ratio of (all-Z)-5,8,11,14,17-eicosapentaen-1-ol:(all-Z)-4,7,10,13,16,19-docosahexaen-1-ol from 3:1 to 1:3.

20. A lipid composition comprising at least a pro-drug of omega-3 polyunsaturated alcohols, wherein the pro-drug of omega-3 polyunsaturated alcohols comprise at least pro-drugs of (all-Z)-5,8,11,14,17-eicosapentaen-1-ol and (all-Z)-4,7,10,13,16,19-docosahexaen-1-ol.

21. A lipid composition according to claim 20, wherein the pro-drugs of omega-3 polyunsaturated alcohols are present in a concentration of least 30% by weight of the total lipid content of the composition.

22. A lipid composition according to claim 20, wherein the pro-drugs of omega-3 polyunsaturated alcohols are present in a concentration of least 50% by weight of the total lipid content of the composition.

23. A lipid composition according to claim 20, wherein the pro-drugs of omega-3 polyunsaturated alcohols are present in a concentration of least 70% by weight of the total lipid content of the composition.

24. A lipid composition according to claim 20, wherein the pro-drugs of omega-3 polyunsaturated alcohols are present in a concentration of least 80% by weight of the total lipid content of the composition.

25. A lipid composition according to claim 20, wherein the pro-drugs of (all-Z) 5,8,11,14,17-eicosapentaen-1-ol and (all-Z)-4,7,10,13,16,19-docosahexaen-1-ol comprise at least 20% by weight of the pro-drugs of omega-3 polyunsaturated alcohols.

26. A lipid composition according to claim 25, wherein the pro-drugs of (all-Z) 5,8,11,14,17-eicosapentaen-l-ol and (all-Z)-4,7,10,13,16,19-docosahexaen-1-ol comprise at least 40% by weight of the pro-drugs of omega-3 polyunsaturated alcohols.

27. A lipid composition according to claim 26, wherein the pro-drugs of (all-Z) 5,8,11,14,17-eicosapentaen-l-ol and (all-Z)-4,7,10,13,16,19-docosahexaen-1-ol comprise at least 70% by weight of the pro-drugs of omega-3 polyunsaturated alcohols.

28. A lipid composition according to claim 27, wherein the pro-drugs of (all-Z) 5,8,11,14,17-eicosapentaen-1-ol and (all-Z)-4,7,10,13,16,19-docosahexaen-1-ol comprise at least 80% by weight of the pro-drugs of omega-3 polyunsaturated alcohols.

29. A lipid composition according to any one of claims 20 to 24, wherein the pro-drugs of omega-3 polyunsaturated alcohols comprise 5% to 95% of a pro-drug of (all-Z) 5,8,11,14,17-eicosapentaen-1-ol by weight of the total lipid content in the composition.

30. A lipid composition according to claim 29, wherein the pro-drugs of omega-3 polyunsaturated alcohols comprise 40% to 55% of a pro-drug of (all-Z) 5,8,11,14,17-eicosapentaen-1-ol by weight of the total lipid content in the composition.

31. A lipid composition according to claim 20, wherein the pro-drugs of omega-3 polyunsaturated alcohols comprise 5% to 95% of a pro-drug of (all-Z)-4,7,10,13,16,19-docosahexaen-1-ol by weight of the total lipid content in the composition.

32. A lipid composition according to claim 31, wherein the pro-drugs of omega-3 polyunsaturated alcohols comprise 30% to 60% of a pro-drug of (all-Z)-4,7,10,13,16,19-docosahexaen-l-ol by weight the total lipid content in the composition.

33. A lipid composition according to claim 20, wherein the pro-drugs of omega-3 polyunsaturated alcohols comprise pro-drugs of (all-Z) 5,8,11,14,17-eicosapentaen-1-ol and (all-Z)-4,7,10,13,16,19-docosahexaen-l-ol in a weight ratio of pro-drug of (all-Z) 5,8,11,14,17-eicosapentaen-1-ol: pro-drug of (all-Z)-4,7,10,13,16,19-docosahexaen-1-ol from 99:1 to 1:99.

34. A lipid composition according to claim 33, wherein the pro-drugs of omega-3 polyunsaturated alcohols comprise pro-drugs of (all-Z)-5,8,11,14,17-eicosapentaen-1-ol and (all-Z)-4,7,10,13,16,19-docosahexaen-1-ol in a weight ratio of pro-drug of (all-Z) 5,8,11,14,17-eicosapentaen-1-ol: pro-drug of (all-Z)-4,7,10,13,16,19-docosahexaen-l-ol from 10:1 to 1:10.

35. A lipid composition according to claim 34, wherein the pro-drugs of omega-3 polyunsaturated alcohols comprise pro-drugs of (all-Z) 5,8,11,14,17-eicosapentaen-1-ol and (all-Z)-4,7,10,13,16,19-docosahexaen-l-ol in a weight ratio of pro-drug of (all-Z) 5,8,11,14,17-eicosapentaen-l-ol: pro-drug of (all-Z)-4,7,10,13,16,19-docosahexaen-l-ol from 5:1 to 1:5.

36. A lipid composition according to claim 35, wherein the pro-drugs of omega-3 polyunsaturated alcohols comprise pro-drugs of (all-Z) 5,8,11,14,17-eicosapentaen-l-ol and (all-Z)-4,7,10,13,16,19-docosahexaen-1-ol in a weight ratio of pro-drug of (all-Z) 5,8,11,14,17-eicosapentaen-1-ol: pro-drug of (all-Z)-4,7,10,13,16,19-docosahexaen-l-ol from 3:1 to 1:3.

37. A lipid composition according to claim 35, wherein the pro-drugs of omega-3 polyunsaturated alcohols comprise pro-drugs of (all-Z) 5,8,11,14,17-eicosapentaen-l-ol and (all-Z)-4,7,10,13,16,19-docosahexaen-1-ol in a weight ratio of pro-drug of (all-Z) 5,8,11,14,17-eicosapentaen-1-ol: pro-drug of (all-Z)-4,7,10,13,16,19-docosahexaen-l-ol from 1:2 to 2:1.

38. A lipid composition according to claim 35, wherein the pro-drugs of omega-3 polyunsaturated alcohols comprise pro-drugs of (all-Z) 5,8,11,14,17-eicosapentaen-l-ol pro-drug and (all-z)-4,7,10,13,16,19-docosahexaen-1-ol pro-drug in a weight ratio of pro-drug of (all-Z) 5,8,11,14,17-eicosapentaen-l-ol: pro-drug of (all-Z)-4,7,10,13,16,19-docosahexaen-l-ol about 1.2.

39. A lipid composition according to claim 1, wherein at least 65% by weight of the omega-3 polyunsaturated alcohols is comprised of (all-Z) 5,8,11,14,17-eicosapentaen-1-o1 and (all-Z)-4,7,10,13,16,19-docosahexaen-l-ol, in a weight ratio of (all-Z) 5,8,11,14,l7-eicosapentaen-1-ol:(all-Z)-4,7,10,13,16,19-docosahexaen-l-ol from 3:1 to 1:3.

40. A lipid composition according to claim 1, wherein at least 70% by weight of the omega-3 polyunsaturated alcohols is comprised of (all-Z) 5,8,11,14,17-eicosapentaen-1-ol and (all-Z)-4,7,10,13,16,19-docosahexaen-l-ol, in a weight ratio of (all-Z) 5,8,11,14,17-eicosapentaen-l-ol:(all-Z)-4,7,10,13,16,19-docosahexaen-l-ol from 1:2 to 2:1.

41. A lipid composition according to claim 1, wherein the lipid composition further comprises an acceptable antioxidant.

42. A lipid composition according to claim 41, wherein the antioxidant is tocopherol.

43. A lipid composition according to claim 1 to 42, formulated for oral administration.

44. A lipid composition according to claim 1, microencapsulated or in a capsule or a sachet.

45. A lipid composition according to claim 44, in a microencapsulated solid dosage form.

46. A lipid composition according to claim 1, formulated to provide a daily amount of omega-3 polyunsaturated alcohols, or pro-drugs thereof, from about 0.1 to 6 g.

47. A lipid composition according to claim 46, formulated to provide a daily amount of omega-3 polyunsaturated lipids, omega-3 polyunsaturated alcohols, pro-drugs of omega-3 polyunsaturated alcohols, or a mixture thereof from about 0.1 to 3.5 g, preferably about 0.5 to 1.7 g.

48. A lipid composition according to claim 1, further comprising omega-3 polyunsaturated alcohols chosen from (all-Z)-6,9,12,15,18-heneicosapentaen-l-ol, (all-Z)-7,10,13,16,19-docosapentaen-l-ol, and (all-Z)-6,9,12,15-octadecatetraen-l-ol, or pro-drug thereof.

49. A lipid composition according to claim 20, wherein said pro-drug of (all-Z) 5,8,11,14,17-eicosapentaen-1-ol is a compound of formula (III), 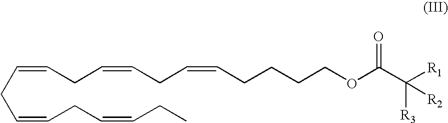
wherein, R₁, R₂, and R₃ are chosen from:
a hydrogen atom,
a C₁-C₂₂ alkyl, and
a C₁-C₂₂ alkenyl with 1 to 6 double bonds in Z or E configuration, wherein the alkyl and alkenyl groups are optionally substituted, or a salt thereof.

50. A lipid composition according to claim 49, wherein said pro-drug of (all-Z) 5,8,11,14,17-eicosapentaen-1-ol is a compound of formula (III), 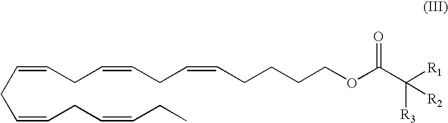
wherein, R₁, R₂, and R₃ are each a hydrogen atom.

51. A lipid composition according to claim 49, wherein said pro-drug of (all-Z) 5,8,11,14,17-eicosapentaen-1-ol is a compound of formula (III), 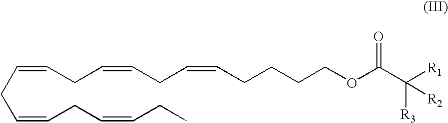
wherein, R₁, R₂, and R₃ are each a methyl group.

52. A lipid composition according to claim 20, wherein said pro-drug of (all-Z)-4,7,10,l3,l6,19-docosahexaen-l-ol is a compound of formula (IV); 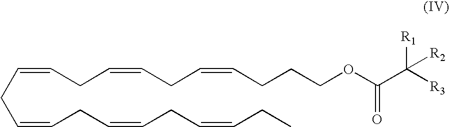
wherein, R₁, R₂, and R₃ are chosen from:
a hydrogen atom,
a C₁-C₂₂ alkyl, and
a C₁-C₂₂ alkenyl with 1 to 6 double bonds in Z or E configuration, wherein the alkyl and alkenyl groups are optionally substituted, or a salt thereof.

53. A lipid composition according to claim 52, wherein said pro-drug of (all-Z)-4,7,10,13,16,19-docosahexaen-l-ol is a compound of formula (IV); 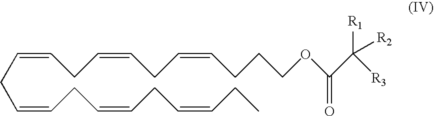
wherein, R₁, R₂, and R₃ are each a hydrogen atom.

54. A lipid composition according to claim 52, wherein said pro-drug of (all-Z)-4,7,10,13,16,19-docosahexaen-l-ol is a compound of formula (IV); 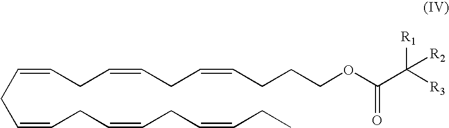
wherein, R₁, R₂, and R₃ are each a methyl group.

55. A lipid composition according to claim 20, wherein said pro-drug of (all-Z) 5,8,11,14,17-eicosapentaen-l-ol is chosen from (5Z,8Z,11Z,14Z,17Z)-eicosapentaen-l-yl pivoloate and (5Z,8Z,11Z,14Z,17Z)-eicosapentaen-1-yl hemisuccinate or a salt thereof.

56. A lipid composition according to claim 20, wherein said pro-drug of (all-Z)-4,7,10,13,16,19-docosahexaen-1-ol is chosen from (4Z,7Z,10Z,13Z,16Z,19Z)-docosahexaen-1-yl pivoloate and (4Z,7Z,10Z,13Z,16Z,19Z)-docosahexaen-1-yl hemisuccinate or a salt thereof.

57. A lipid composition according to claim 1, wherein the lipid composition is a pharmaceutical composition.

58. A lipid or pharmaceutical composition according to claim 1, for use as a medicament or a pharmaceutical, for use in therapy, or for use as a cosmetic skin preparation.

59. A pharmaceutical composition for the treatment of elevated triglyceride levels comprising omega-3 polyunsaturated alcohols in a concentration of at least 80% by weight as compared to the total lipid content of the composition, and wherein at least 70% of the omega-3 polyunsaturated alcohols is comprised of a combination of (all-Z) 5,8,11,14,17-eicosapentaen-1-ol and (all-Z)-4,7,10,13,16,19-docosahexaen-1-ol in a weight ratio of (all-Z) 5,8,11,14,17-eicosapentaen-l-ol:(all-Z)-4,7,10,13,16,19-docosahexaen-1-ol from 1:3 to 3:1.

60. A pharmaceutical composition for the treatment of elevated triglyceride levels comprising omega-3 polyunsaturated alcohols in a concentration of at least 80% by weight as compared to the total lipid content of the composition, and wherein at least 70% of the omega-3 polyunsaturated alcohols is comprised of a combination of (all-Z) 5,8,11,14,17-eicosapentaen-1-ol and (all-Z)-4,7,10,13,16,19-docosahexaen-1-ol in a weight ratio of (all-Z) 5,8,11,14,17-eicosapentaen-1-ol:(all-Z)-4,7,10,13,16,19-docosahexaen-1-ol from 1 to 1.5.

61. A lipid composition according to claim 1, for use as a food or a “lifestyle” supplement.

62-68. (canceled)

69. A method of treatment and/or prevention of hypertriglyceridemia (HTG), dyslipidemia, hypertension, hypercholesteremia, post-myocardial infarction (MI) or depression, heart failure, cardiac arrhythmias or atrial fibrillation, high risk patients with homeostasis, IgA Nephropathy, vascular diseases and/or atherosclerotic diseases, wherein a therapeutically effective amount of the lipid composition according to claim 1 to is administered to a human or an animal.

70. A method for reducing abnormal triglyceride levels in a patient, preferably reducing triglyceride levels of about 200 to about 499 mg/dl, wherein a therapeutically effective amount of the lipid composition according to claim 1 is administered to a human or an animal.

71. A process for manufacture of a lipid composition according to claim 1.

72. A process for manufacture of a lipid composition according to claim 1, wherein said lipid composition is prepared from a vegetable, a microbial and/or an animal source.

73. A process for manufacture of a lipid composition according to claim 1, wherein said lipid composition is prepared from a marine oil.

74. A process for manufacture of a lipid composition according to claim 73, wherein said lipid composition is prepared from a fish oil or a krill oil.

75. A process for manufacture of a lipid composition according to claim 1, wherein
the raw material is a up-concentrated mixed-fatty acid composition comprising at least 50% of weight of omega-3 fatty acid esters, comprising esters of at least the omega-3 C 20:5 and C 22:6 acids, and
the esters of the omega-3 C 20:5 and C 22:6 acids are reduced to polyunsaturated alcohols, by using a reagent that transfer a hydride from boron or aluminium to the carbonyl compound.

76. A process for the manufacture of a lipid composition according to claim 52, wherein
the raw material is a up-concentrated mixed-fatty acid composition comprising at least 50% of weight of omega-3 fatty acid esters, comprising esters of at least the omega-3 C 20:5 and C 22:6 acids, and
the esters of the omega-3 C 20:5 and C 22:6 acids are reduced to polyunsaturated alcohols, by using a reagent that transfer a hydride from boron or aluminium to the carbonyl compound.
the resulting omega-3 C 20:5 and C 22:6 alcohols are acylated.

77. A process for manufacture of a lipid composition according to claim 75, wherein said reagent is selected from the group consisting of Lithium aluminium hydrides, such as LiA1H₄, LiA1H₂(OCH₂CH₂OCH₃) or LiA1H[OC(CH₃)₃]₃, and boron hydrides such as: LiBH₄, or Ca(BH₄)₂.

78. A compound of formula (III): 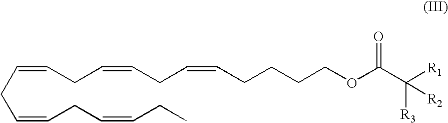
wherein, R₁, R₂, and R₃ are chosen from:
a hydrogen atom,
a C₁-C₂₂ alkyl, and
a C₁-C₂₂ alkenyl with 1 to 6 double bonds in Z or E configuration, wherein the alkyl and alkenyl groups are optionally substituted, or a salt thereof, with the proviso that R₁, R₂, and R₃ are not each simultaneously a hydrogen atom.

79. A compound of formula (IV): 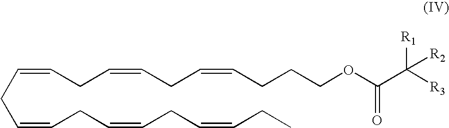
wherein R₁, R₂, and R³ are chosen from:
a hydrogen atom,
a C₁-C₂₂ alkyl, and
a C₁-C₂₂ alkenyl with 1 to 6 double bonds in Z or E configuration, wherein the alkyl and alkenyl groups are optionally substituted, or a salt thereof, with the proviso that R₁, R₂, and R₃ are not each simultaneously a hydrogen atom.

80. A compound according to claim 78 of formula (VIII)

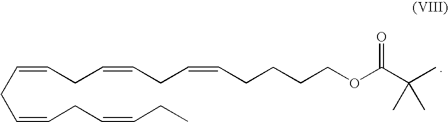

81. A compound according to claim 78 of formula (X) 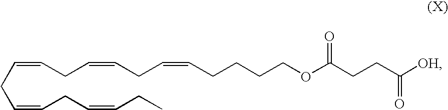
or a salt thereof.

82. The compound according to claim 81, where in the salt is chosen from 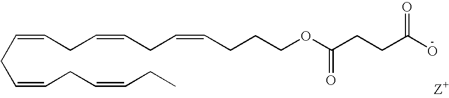
wherein Z⁺ is selected from the group consisting of Li⁺, Na⁺, K⁺, NH₄⁺, 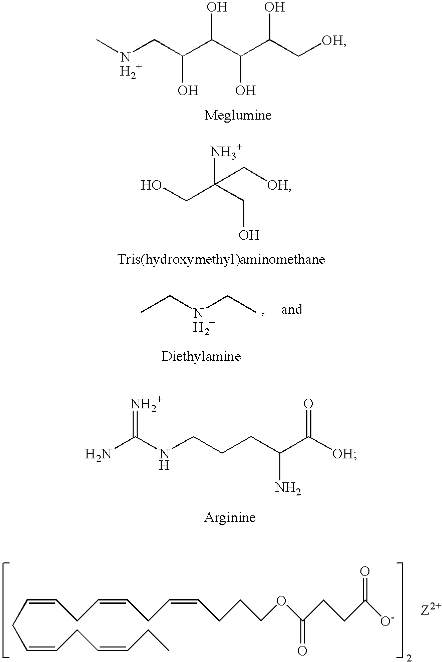
wherein Z²⁺ is selected from the group consisting of Mg^{2÷}, Ca²⁺, 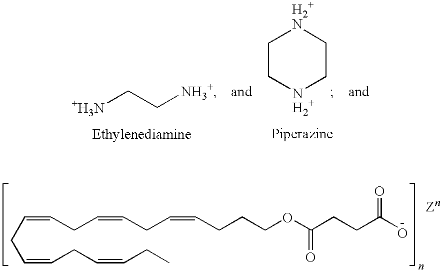
Zⁿ⁺ is

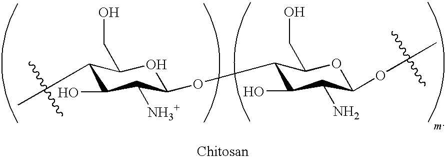

83. A compound according to claim 78 of the following formula:

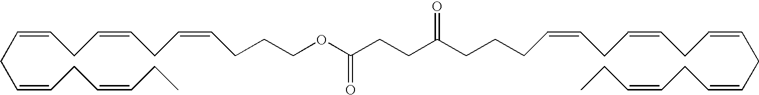

84. A compound according to claim 79 of formula (IX)

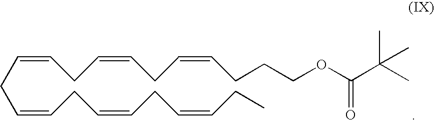

85. A compound according to claim 79 of formula (XI) 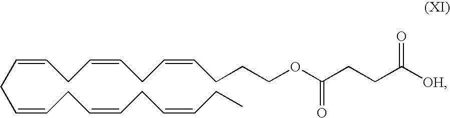
or a salt thereof.

86. The compound according to claim 85, where in the salt is chosen from 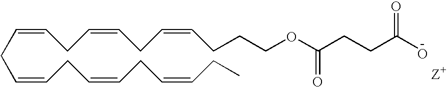
wherein Z⁺ is selected from the group consisting of Li⁺, Na⁺, K⁺, NH₄⁺, 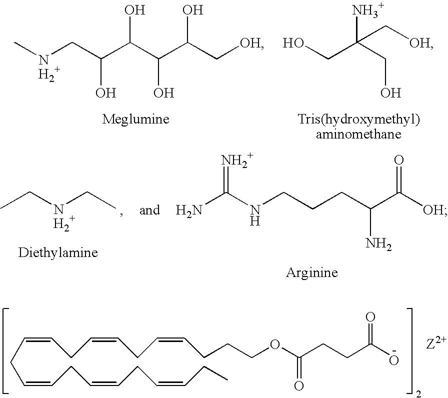
wherein Z²⁺ is selected from the group consisting of Mg²⁺, Ca²⁺,

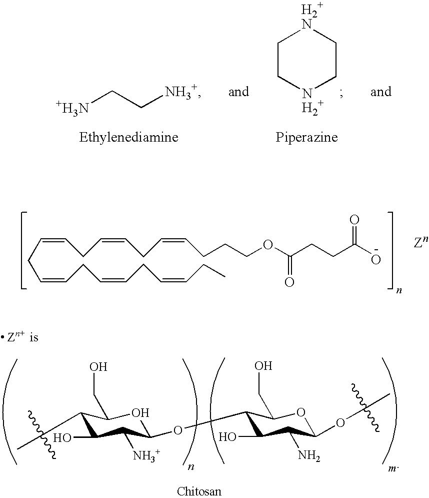

87. A compound according to claim 79 of the following formula:

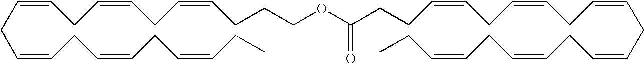

88. A compound of formula (V): 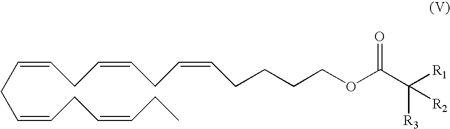
wherein R₁, R₂, and R₃ are chosen from:
a hydrogen atom,
a C₁-C₂₂ alkyl, and
a C₁-C₂₂ alkenyl with 1 to 6 double bonds in Z or E configuration, wherein the alkyl and alkenyl groups are optionally substituted, or a salt thereof.

89. A compound according to claim 88 of the following formula: 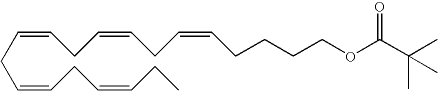
(all-Z)-9,12,15-octadecatrien-l-yl pivaloate ester.

90. A compound according to claim 88 of the following formula: 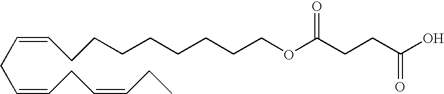
(all-Z)-9,12,15-octadecatrien-l-yl hemisuccinate, or a salt thereof.

91. The compound according to claim 90, where in the salt is chosen from 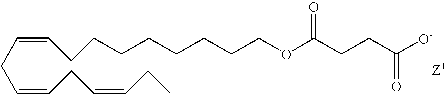
wherein Z⁺ is selected from the group consisting of Li⁺, Na⁺, K⁺, NH₄⁺, 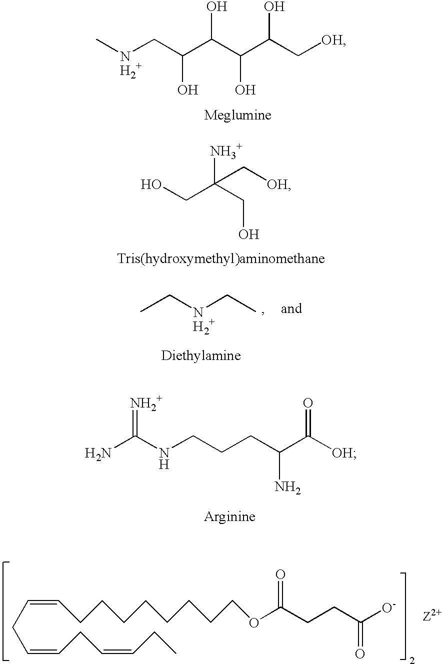
wherein Z²⁺ is selected from the group consisting of Mg²⁺, Ca²⁺, 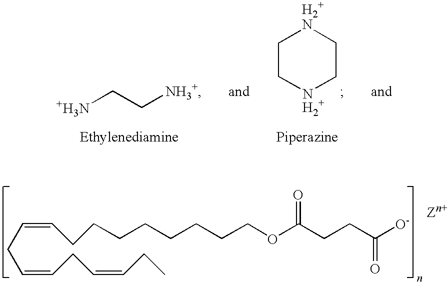
Zⁿ⁺ is 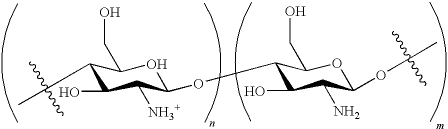
Chitosan.

92. A compound according to claim 88 of the following formula: [chemical structure]
